SEQUENCE LISTING (19 sequences)

SEQ ID NO: 1 - protein, 14 aa, synthetic construct
NFQGVQNRFVFGTP

SEQ ID NO: 2 - protein, 16 aa, synthetic construct
MENAELDVPIQSVFTR

SEQ ID NO: 3 - protein, 11 aa, synthetic construct
NTDNIYPESSC

SEQ ID NO: 4 - protein, 8 aa, synthetic construct
PYLGYVFK

SEQ ID NO: 5 - protein, 18 aa, synthetic construct
MQTVAQLFKTVSSLSLST

SEQ ID NO: 6 - protein, 19 aa, synthetic construct
HSPDIQLQKGLTFEPIQIK

SEQ ID NO: 7 - protein, 16 aa, synthetic construct
STITQPYKTLNNARSP

SEQ ID NO: 8 - protein, 15 aa, synthetic construct
RPGPSPEGTGQSYNY

SEQ ID NO: 9 - protein, 16 aa, synthetic construct
MENAELDPPYLGYVSK

SEQ ID NO: 10 - protein, 20 aa, synthetic construct
TGQSYNQYSQRPYLGVYVFK

SEQ ID NO: 11 - protein, 10 aa, synthetic construct
LYGQTPLETL

SEQ ID NO: 12 - protein, 11 aa, synthetic construct
ELADSPALEIG

SEQ ID NO: 13 - protein, 21 aa, synthetic construct
LYGQTPLETLELADSPALEIG

SEQ ID NO: 14 - protein, 14 aa, synthetic construct
VSGNTVEYALPTLE

SEQ ID NO: 15 - protein, 18 aa, synthetic construct
LDSPTAPTVQSTALTWRP

SEQ ID NO: 16 - protein, 17 aa, synthetic construct
LDGSAPGPLYTGSALDF

SEQ ID NO: 17 - protein, 11 aa, synthetic construct
GSEGVRSGRSG

SEQ ID NO: 18 - protein, 14 aa, synthetic construct
QPQPLPSPGVGGKN

SEQ ID NO: 19 - protein, 11 aa, synthetic construct
KYTLNPVIDAS

(19) United States
(12) Patent Application Publication
(10) Pub. No.: US 2025/0276999 A1
(43) Pub. Date: Sep. 4, 2025

(54) PEPTIDES FOR PROMOTING HAIR FOLLICLE NEOGENESIS, AND PREVENTION/MITIGATION OF UNTOWARD EFFECTS OF RADIATION TREATMENT

(71) Applicants: Trustees of Tufts College, Medford, MA (US); The Henry M. Jackson Foundation for the Advancement of Military Medicine, Inc., Bethesda, MD (US)

(72) Inventors: Ira M. Herman, Boston, MA (US); Thomas N. Darling, Bethesda, MD (US); Regina M. Day, Bethesda, MD (US)

(21) Appl. No.: 18/857,555

(22) PCT Filed: Apr. 18, 2023
(86) PCT No.: PCT/US2023/065920

§ 371 Date: Oct. 17, 2024

Related U.S. Application Data
(60) Provisional application No. 63/363,241, filed on Apr. 19, 2022.

(51) Int. Cl. C07K 7/06; A61K 38/00; C07K 7/08; C07K 14/00

(52) U.S. Cl. CPC C07K 7/06; C07K 7/08; C07K 14/001; A61K 38/00

(57) ABSTRACT

The disclosed subject matter relates to peptides that are derived from treating the extracellular matrix (ECM) with collagenase and methods of using the peptides thus derived. The peptides may be used for applications including, but not limited to, promoting hair follicle neogenesis, coloration, and mitigating negative effects of radiation treatment.

### CROSS-REFERENCE TO RELATED APPLICATIONS

This application claims the benefit of U.S. Provisional Application 63/363,241 with filing date of Apr. 19, 2022, the content of which is incorporated herein by reference in its entirety.

### STATEMENT REGARDING FEDERALLY SPONSORED RESEARCH OR DEVELOPMENT

This invention was made with government support under HU0001-21-2-0061, HU0001-21-2-0035, and HU0001-17-2-0009 awarded by the Uniformed Services University of the Health Sciences. The government has certain rights in the invention.

### FIELD

The field of the invention relates to peptides that are derived from treating the extracellular matrix (ECM) with collagenase and methods of using the peptides thus derived³². The peptides may be used for applications including, but not limited to, promotion of hair follicle neogenesis, and mitigating negative effects of radiation treatment, including but not limited to prevention of cutaneous injury, skin ulceration, hair loss while promoting wound healing, hair growth and coloration.

### REFERENCE TO AN ELECTRONIC SEQUENCE LISTING

The contents of the electronic sequence listing (166118.01300.xml; Size: 17,505 bytes; and Date of Creation: Apr. 12, 2023) is herein incorporated by reference in its entirety.

### BACKGROUND

Hair represents a defining characteristic of mammals and is critical to animals and humans for thermal regulation, nonverbal communication, social acceptance and beatification. Hair morphogenesis takes place mostly in utero with no new hair follicles formation in adulthood. The significance of this loss of hair follicle neogenesis becomes particularly apparent following age-related hair loss, hair loss accompanying skin conditions, clinical interventions, thermal injury and cutaneous wound healing. Human wound healing typically results in a scarring, which can also give rise to disfigured skin, which lacks hair follicles. Hair loss due to wounding or age can cause adverse psychological sequalae. Current therapies to treat hair loss depend upon the presence of existing hair follicles; however, there are currently no therapies available, or there is a dearth of therapies or treatment options for promoting hair growth by stimulation of hair follicle neogenesis, i.e. the genesis of new hair follicles when existing follicles have been lost or have been destroyed by clinical intervention, thermal injury or radiation damage.

Dermal papilla cells regulate hair follicle development and growth. Methods to promote dermal papilla cells, especially the trichogenicity of these cells, is paramount to establishing hair follicle neogenesis. Unfortunately, maintaining dermal papilla cells' inductive capacity is difficult in culture, as evident by their gradual loss of alkaline phosphatase activity in culture. Novel methods to maintain neogenesis of dermal papilla cell hair follicles are an unmet need across the wound care continuum, including hair loss that is the result of radiation therapy.

Radiation therapy is a mainstay of cancer treatment. Unfortunately, almost all patients undergoing radiation therapy will develop cutaneous tissue-associated radiation damage or radiotherapy-induced radiation dermatitis. Damage to underlying normal tissues limits the use of radiotherapy for cancer treatment. In all patients, the post-irradiation skin is permanently changed and cannot repair itself from wounds, cuts, or surgical interventions. Radiation damage is a painful and debilitating effect of this treatment which can impact quality of life and treatment outcomes. Radiation can cause persistent damage to the skin, including permanent hair loss and ulceration. Current standard of care for radiotherapy-induced radiation dermatitis includes AQUAPHOR, mineral oil, and lanolin alcohol. Strategies under investigation to prevent or mitigate radiation dermatitis include some clinical trials, treated wound dressings, anti-inflammatory agents, antibiotics, laser treatment or skin grafts. Despite these attempts at mitigating or preventing radiation-induced dermatitis and the accompanying untoward effects observed in all patients, there remains a global and an urgent, unmet clinical need aimed at prevention.

In view of the foregoing, it would be desirable to provide new methods to safely and efficiently promote the generation of new hair follicles in a subject exhibiting hair loss due to natural causes such as e.g., age and/or genetic diseases, thermal injury, or radiation injury (e.g., due to radiation therapy or other radiation-related injury). It also would be desirable to provide new methods for prevention or mitigation of additional negative effects of radiation treatment such as radiation dermatitis.

### SUMMARY

Provided herein are methods of treating subjects with bioactive peptides. The disclosed methods may be practiced in order to stimulate hair follicle neogenesis and resultant hair growth in a subject in need thereof. The disclosed methods also may be practiced in order to prevent and/or mitigate and/or treat the negative effects of radiation treatment. The methods disclosed herein may be used to treat hair loss due, for example, natural causes such as e.g., age and/or genetic diseases, thermal injury, or radiation injury (e.g., due to radiation therapy or other radiation-related injury).

In some embodiments, the disclosed methods may be performed for treating a subject having hair loss, for example, by promoting hair follicle neogenesis in the subject. The method may comprise administering to the subject an effective amount of a peptide that is derived from treating the extracellular matrix (ECM) with collagenase, such as a bacterial collagenase, for treating hair loss in the subject. In some embodiments of the disclosed methods for treating hair loss, the peptide comprises, consists essentially of, or consists of the amino acid sequence of any of SEQ ID NOs: 1-19, or any combinations thereof. The subject may have hair loss, which is attributed to the loss or death of hair follicles, such as loss or death of hair follicles attributed to age, loss or death of hair follicles attributed to testosterone levels in the subject (e.g., androgenetic alopecia), and/or loss or death of hair follicles attributed to thermal injury and/or radiation injury. The disclosed methods may promote hair follicle neogenesis in a subject treated with the disclosed methods.

In other embodiments, the disclosed methods may be performed to prevent and/or mitigate the negative effects of radiation therapy in a subject who has undergone radiation therapy or in a subject who will undergo radiation therapy. The negative effects of radiation therapy that are mitigated by the disclosed methods may include dermatitis and hair follicle loss or death or mitigation of skin ulceration, erythema, fibrosis, or inflammation. The method may comprise administering to the subject an effective amount of a peptide that is derived from treating the extracellular matrix (ECM) with collagenase, such as a bacterial collagenase, thereby mitigating the negative effects of radiation therapy in the subject. In some embodiments of the methods for mitigating the negative effects of radiation therapy, the peptide comprises, consists essentially of, or consists of the amino acid sequence of any of SEQ ID NOs: 1-19, or a combination thereof. The peptide may be administered to the subject before the subject is administered radiation therapy and/or the peptide may be administered to the subject after the subject is administered radiation therapy. As such, the disclosed methods may include administered radiation therapy to a subject. The subject may be undergoing radiation therapy to treat cancer, and as such, a suitable subject for the disclosed methods may include a subject having cancer. The subject may have been exposed to radiation accidentally or due to a military conflict involving the deployment of radiation. In the disclosed methods, the subject may be administered a dose of radiation that causes dermatitis and/or the loss or death of hair follicles, and the subject may be administered an effective amount of a peptide for treating the dermatitis and/or an effective amount of a peptide for treating the hair loss, for example, by stimulating hair follicle neogenesis. In the disclosed methods, subjects may be administered an amount of peptide that is effective for promoting neogenesis of hair follicles and preventing and/or mitigating stem cell senescence in the skin, prevention or mitigation of cutaneous injury and/or ulceration.

The peptides utilized in the disclosed methods typically are derived by treating the ECM with a collagenase, such as a bacterial collagenase. The peptides may be resistant to degradation after the peptides are administered to a treatment site in a subject, such as the scalp of the subject, a site on the subject that has been administered radiation therapy, and/or a wound bed of the subject. Treatment sites such as wound beds are known to comprise proteases, and prior attempts to develop advanced, protein-based neogenesis therapeutics for treating wound beds have failed at least partially because the proteases present in wound beds will degrade any bioactive protein that is delivered to wound beds. For these reasons, the peptides utilized in the disclosed methods may be resistant to degradation by proteases, including proteases which are present in wound beds.

Also disclosed herein are compositions comprising the disclosed peptides, which compositions are formulated for use in the methods disclosed herein. Compositions disclosed herein may include compositions formulated for topical administration at a treatment site on a subject for treating hair loss, such as a scalp of the subject, and/or on a treatment site of the subject for preventing and/or mitigating the negative effects of radiation therapy that is administered at the treatment site. The disclosed compositions may be formulated for topical administration at a wound bed and/or for topical administration to a graft or radiation wound, for example.

### BRIEF DESCRIPTION OF THE DRAWINGS

The present technology can be better understood by reference to the following drawings. The drawings are merely exemplary to illustrate certain features that may be used singularly or in combination with other features and the present technology should not be limited to the embodiments shown.

FIG. 1. Dose response of TSN6 on alkaline phosphatase activity as a marker of trichogenicity of scrambled (left) and active (right) TSN6 peptides.

FIG. 2. Hair follicle neogenesis in vivo. TSN6 peptide patch assay counts of number of hair follicles for 100 μM of scrambled peptide (white bar) and TSN6 peptide (black bar). TSN6 induces a two-fold increase in hair follicle formation. One patch equals 250 spheroids (4000 hDP cells/spheroid and ˜50,000 mouse epidermal aggregates). N=3 patches for each peptide. Graph is mean±standard deviation.

FIG. 3. TSN6 increases human hair follicle formation in grafted DECs. Immunohistochemical staining for human HLA in TSN6-scrambled (left most panel) and TSN6-grafts from 2 mice showing hair follicle anatomy. Mice were euthanized 12 weeks after grafting and grafts were analyzed for human cells by immunohistochemical staining for human leukocyte antigen (red).

FIG. 4. TSN6 stimulates epidermal proliferation in vivo. Ki-67⁺ cells in epidermis of grafts (cells/mm) with 1 μM of scrambled (white bar) or active (black bar) TSN6 peptides. P=0.08

FIG. 5. Mitigation of radiation dermatitis by TSN6 peptide administration in mice. Female C57BL/6 mice (12-14 weeks of age) were exposed to 16.5 Gy thoracic X-ray irradiation. Mice received either vehicle (saline) or TSN6 peptide (5 μg/day) by subcutaneous injection on days 2, 7 and 10 post-irradiation. Mice were scored for: A) hair loss; B) scale; C) erythema; or D) ulceration at the indicated times post-irradiation. Combined scores are shown in E). Data indicate means±SEM, N=4 animals/group.

FIG. 6. Histology of dorsal thoracic region 83 days following X-ray irradiation. Female C57BL/6 mice (12-14 weeks of age) were exposed to 16.5 Gy thoracic X-ray irradiation. Mice received either sham irradiation treatment (A), irradiation plus vehicle (saline) (B), or irradiation plus TSN6 peptide (5 μg/day) by subcutaneous injection on days 2, 7 and 10 post-irradiation (C). Skin sections were obtained 83 days post-irradiation after euthanization. Skin was formalin fixed and paraffin embedded prior to staining with Hematoxylin & Eosin. Sections are cranial to caudal from left to right. Arrows indicate area of irradiation. Representative images are shown.

FIG. 7. Mitigation of combined injury by TSN6 peptide administration in mice. Female C57BL/6 mice (12-14 weeks of age) were exposed to 16.5 Gy thoracic X-ray irradiation and were then given a 9/16 inch wound. Mice received either vehicle (saline) or TSN6 peptide (5 μg/day) by subcutaneous injection on days 2, 7 and 10 post-irradiation. Mice were scored for: A) wound closure; B) hair loss; C) scale; D) erythema; or E) ulceration at the indicated times post-irradiation. Combined scores are shown in F). Data indicate means±SEM, N=4 animals/group.

FIG. 8. Histology of dorsal thoracic region at 83 days following combined injury. Female C57BL/6 mice (12-14 weeks of age) were exposed to 16.5 Gy thoracic X-ray irradiation and then received a 9/16 inch wound. Mice received either sham irradiation (A); irradiation plus vehicle (saline) (B), or irradiation plus TSN6 peptide (5 μg/day) by subcutaneous injection on days 2, 7 and 10 post-irradiation (C). Skin sections were obtained at 83 days post-irradiation after euthanization. The area of the wound was marked with black ink on the surface, visible on the top of the sections. Skin was formalin fixed and paraffin embedded prior to staining with Hematoxylin & Eosin. Sections are cranial to caudal from left to right. Arrows indicate area of wound. Representative images are shown.

FIG. 9. HGF administration on radiation dermatitis in mice. Female C57BL/6 mice (12-14 weeks of age) were exposed to 16.5 Gy thoracic X-ray irradiation. Mice received either vehicle (saline) or HGF (3.3 μg/day) by subcutaneous injection on days 2, 7 and 10 post-irradiation. Mice were scored for: A) hair loss; B) scale; C) erythema; or D) ulceration at the indicated times post-irradiation. Combined scores are shown in E). Data indicate means±SEM, N=4 animals/group.

FIG. 10. Histology of dorsal thoracic region at 83 days following X-ray irradiation. Female C57BL/6 mice (12-14 weeks of age) were exposed to 16.5 Gy (0.77 Gy/min) thoracic X-ray irradiation. Mice received either sham irradiation (A); irradiation plus vehicle (saline) (B), or irradiation plus HGF peptide (3.3 μg/day) by subcutaneous injection on days 2, 7 and 10 post-irradiation (C). Skin sections were obtained at 83 days post-irradiation after euthanization. Skin was formalin fixed and paraffin embedded prior to staining with Hematoxylin & Eosin. Sections are cranial to caudal from left to right. Arrows indicate area of irradiation. Representative images are shown.

FIG. 11. Mitigation of combined injury by TSN6 peptide administration in mice. Female C57BL/6 mice (12-14 weeks of age) were exposed to 16.5 Gy (0.77 Gy/min) thoracic X-ray irradiation and were then given a 9/16 inch wound. Mice received either vehicle (saline) or HGF (3.3 μg/day) by subcutaneous injection on days 2, 7 and 10 post-irradiation. Mice were scored for: A) wound closure; B) hair loss; C) scale; D) erythema; or E) ulceration at the indicated times post-irradiation. Combined scores are shown in F). Data indicate means±SEM, N=4 animals/group.

FIG. 12. Histology of dorsal thoracic region at 83 days following combined injury. Female C57BL/6 mice (12-14 weeks of age) were exposed to 16.5 Gy (0.77 Gy/min) thoracic X-ray irradiation and then received a 9/16 inch wound. Mice received either sham irradiation (A), irradiation plus vehicle (saline) (B), or irradiation plus HGF (3.3 μg/day) by subcutaneous injection on days 2, 7 and 10 post-irradiation (C). Skin sections were obtained at 83 days post-irradiation after euthanization. The wound area was marked with black ink on the surface of the skin, visible in most section. Skin was formalin fixed and paraffin embedded prior to staining with Hematoxylin & Eosin. Sections are cranial to caudal from left to right. Arrows indicate area of wound. Representative images are shown.

FIG. 13. Images of hair regrowth and repair following irradiation. Treatment with TSN6 improves radiation dermatitis and regrows pigmented hair in mice. Images of mice treated with vehicle (top panels) or TSN6 (bottom panels) A) 57 days, B) 71 days and C) 79 days post-irradiation.

FIG. 14. A. Graft presence was evaluated at the end of the experiment (10 weeks) in Nude mice grafted with active peptide 10 μM (TSN18) (n=5), scrambled peptide 10 μM (n=5) and vehicle (n=5) treated dermal-epidermal constructs; B. Graft size was measured at 10 weeks in TSN18 (n=4), scrambled (n=3) peptide 10 μM each and vehicle (n=4) treated groups, ns: Not significant

FIG. 15. Injection of active but not scrambled TSN18 peptide decreased the severity of our aggregate radiation dermatitis scores of the mouse skin by nearly 50%; 5 mice per group

### DETAILED DESCRIPTION

Other objects, features, and advantages of the technology provided herein will become apparent from the following detailed description. It should be understood, however, that the detailed description and the specific examples, while indicating preferred embodiments of the technology provided herein, are given by way of illustration only, since various changes and modifications within the spirit and scope of the technology provided herein will become apparent to those skilled in the art from this detailed description.

### Peptides for Use in the Disclosed Methods

Peptides that are suitable for use in the disclosed methods may be obtained by treating the extracellular matrix of tissue with a collagenase that digests protein present in the ECM, and releases peptides. Suitable collagenases may include bacterial collagenases. Peptide that are suitable for use in the disclosed methods are described in U.S. Pat. No. 10,485,846, and Sheets et al, 2016 PLOS One, the content of which is incorporated herein by reference in its entirety.

Suitable peptides for the disclosed methods may include peptides comprising, consisting essentially of, or consisting of any of SEQ ID NOs: 1-19, or combinations thereof, as follow:
TNS1, 14 aa, NFQGVQNRFVFGTP (SEQ ID NO:1) Thrombospondin-1 Laminin G-like domain;
TSN2, 16 aa, MENAELDVPIQSVFTR (SEQ ID NO:2) Thrombospondin-1 N-terminal domain;
TSN3, 11 aa, NTDNIYPESSC (SEQ ID NO:3) Multimerin EGF-like domain;
TSN4, 8 aa, PYLGYVFK (SEQ ID NO:4) Multimerin Clq domain;
TSN5, 18 aa, MQTVAQLFKTVSSLSLST (SEQ ID NO:5) Multimerin-1 Coiled coil domain;
TSN6, 19 aa, HSPDIQLQKGLTFEPIQIK (SEQ ID NO:6) Multimerin-1 Coiled coil domain;
TSN7, 16 aa, STITQPYKTLNNARSP (SEQ ID NO:7) Fibronectin Heparin-binding domain;
TSN8, 16 aa, RPGPSPEGTGQSYNYR (SEQ ID NO:8) Fibronectin Fibrin-binding 2 domain;
TSN9, 16 aa, MENAELDPPYLGYVFK (SEQ ID NO:9) Combination of Thrombospondin and multimerin peptides;
TSN10, 20 aa, TGQSYNQYSQRPYLGVYVFK (SEQ ID NO:10) Combination of Thrombospondin and multimerin peptides
TSN11, 10 aa, LYGQTPLETL (SEQ ID NO:11) TGF-β-induced protein: Fas1/3 domain
TSN12, 11 aa, ELADSPALEIG (SEQ ID NO:12) TGF-.beta.-induced protein: N-terminal domain
TSN13, 21 aa, LYGQTPLETLELADSPALEIG (SEQ ID NO:13) Combination of 11 and
TSN14, 14 aa, VSGNTVEYALPTLE (SEQ ID NO:14) Tenascin C Fibronectin Type III domain 14
TSN15, 18 aa, LDSPTAPTVQSTALTWRP (SEQ ID NO:15) Tenascin C Fibronectin III domain 15
TSN16, 17 aa, LDGSAPGPLYTGSALDF (SEQ ID NO:16) Collagen (VI) Alpha-3 VWFA domain 3
TSN17, 11 aa, GSEGVRSGRSG (SEQ ID NO:17) Collagen (VI) Alpha-3 VWFA domain 6
TSN18, 14 aa, QPQPLPSPGVGGKN (SEQ ID NO:18) Combination of non-helical Collagen (VI) Alpha-3 chain regions;
TSN19, 11 aa, KYTLNPVIDAS (SEQ ID NO: 19) Combination of fibronectin Type-III 14 domain of fibronectin.

In some embodiments, a peptide includes an amino acid sequence selected from the group consisting of SEQ ID NOs: 1-19, and the peptide comprises 100, 95, 90, 85, 80 75, 70, 65, 60, 55, 50, 45, 40, 35, 30, 25, 20, or fewer amino acids. Thus peptide for use in the disclosed methods can comprise, consist essentially of, or consist of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 amino acids.

In some embodiments, a peptide includes an amino acid sequence selected from the group consisting of SEQ ID NOs: 1-19, and the peptide does not comprise a full-length protein from which the peptide was derived. In some of such embodiments, the peptide comprises not more than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9 or 8 contiguous amino acid residues of the protein from which the peptide was derived.

In some embodiments of the disclosed methods, a subject is administered a combinatorial peptide. As used herein, the term “administering” an agent, such as the therapeutic peptides described herein to an animal or cell, is intended to refer to dispensing, delivering or applying the substance to the intended target. The term “administering” is intended to refer to contacting or dispensing, delivering or applying the therapeutic agent to a subject by any suitable route for delivery of the therapeutic agent to the desired location in the animal (e.g., a subject).

A combinatorial peptide is a peptide that comprises the amino acid sequences of a combinations of peptides, for example, a peptide that comprises the amino acid sequence of two or more of the peptides disclosed herein, such as a fusion of the amino acid sequence of two or more of the peptides disclosed herein. A combinatorial peptide typically is not naturally occurring and cannot be produced by treating the ECM with a collagenase. Instead, combinatorial peptides may be produced by chemical synthesis or by expression of a recombinant nucleic acid that encodes the combinatorial peptide, such as with an expression vector comprising a recombinant nucleic acid. Non-limiting examples of combinatorial peptides include peptides that comprise one or more of SEQ ID NOs: 1-8, 11, 12, 14-17. For example, of the peptides disclosed herein, peptides TSN9, TSN10, TSN13, TSN18 and TSN19 (SEQ ID NOs: 9, 10, 13, 18 and 19) are examples of combinatorial peptides (Table 1).

In some embodiments, the peptides can be linked consecutively, with the C-terminal end of one peptide linked via peptide bond to the N-terminal amino acid of another peptide. Additionally or alternatively, in some embodiments, the peptides are linked by one or more linkers that can include one or more amino acids that are not part of the peptides linked in the combinatorial peptide. Peptides can be linked to enzymes, tags or targeting moieties that alter the distribution or localization of the peptides. Peptides may be modified or linked to moieties that alter the half-life or stability of the peptide.

In some embodiments, the peptides can be linked to bioerodible scaffolds-bioinspired microscopic spines able to deliver peptide(s) within/under skin.

In some embodiments, the peptides can be combined with a carrier or excipient. Peptides may be administered as a pharmaceutical composition comprising one or more peptides in combination with one or more pharmaceutically acceptable carriers or excipients. Such compositions may be aqueous solutions, emulsions, creams, ointments, suspensions, gels, liposomal suspensions, and the like. Suitable carriers (excipients) include water, saline, Ringer's solution, dextrose solution, and solutions of ethanol, glucose, sucrose, dextran, mannose, mannitol, sorbitol, polyethylene glycol (PEG), phosphate, acetate, gelatin, collagen, CARBOPOL®, vegetable oils, and the like. One may additionally include suitable preservatives, stabilizers, antioxidants, antimicrobials, and buffering agents, for example, BHA, BHT, citric acid, ascorbic acid, tetracycline, and the like. Cream or ointment bases useful in formulation include lanolin, SILVADENE®, AQUAPHOR®, and the like. Other topical formulations include aerosols, conditioners, bandages and other wound dressings. Alternatively one may incorporate or encapsulate the wound healing peptides in a suitable polymer matrix or membrane, thus providing a sustained-release delivery device suitable for implantation near the site to be treated locally. Other suitable devices for delivering or administering the compositions provided herein include indwelling catheters and devices such as the ALZET® minipump. Ophthalmic preparations may be formulated using commercially available vehicles such as SORBI-CARE®, NEODECADRON®, LACRILUBE®, and the like or may employ topical preparations such as that described in U.S. Pat. No. 5,124,155, incorporated herein by reference.

The peptides described herein may be combined with additional treatments, therapies or therapeutic devices, e.g., provided as a combination therapy. Additional treatments, therapies or therapeutic devices include, but are not limited to, Minoxidil, Finasteride, Baricitinib, spironolactone, dutasteride, corticosteroids, platelet-rich plasma, surgery, laser therapy, microneedeling, the Follica device, the RECELL system, allogeneic cellularized scaffold products including StrataGraft, skin scaffolds including naturally derived or bioengineered scaffolds, bioengineered skin substitutes, skin constructs, skin grafts (split-thickness grafts, full-thickness grafts and composite grafts) and cosmetic procedures. In some embodiments, the peptide may be combined ex-vivo with additional treatments or therapies. By way of example but not limitation, an effective amount of a peptide may be combined with a bioengineered skin construct or substitute, along with media and growth factors useful for inducing differentiation, growth, and survival of the bioengineered skin construct or substitute.

In the disclosed methods, an effective amount of a peptide is administered for treatment, prevention, or mitigation of symptoms (e.g., hair loss). In some embodiments, the effective amount administered in the disclosed methods may be at least 1, 10, or 100 μM or any amount between 1 nM to 100 μM peptide at the site of administration. The effective amount may be administered daily, once or multiple times, or as frequently as needed (e.g., weekly, twice weekly, monthly, etc.).

In some embodiments, the effective amount of the peptide is administered topically. In some embodiments, the effective amount is administered by any effective route, including but not limited to, orally, subcutaneously, subdermal, and/or at the site of a wound bed or lesion. In some embodiments, the effective amount of the peptide is incorporated into topical solutions (including gels, ointments, creams, and suspensions), dressings, patches, follicular units, ex-vivo cells, scaffolds, skin equivalents or transdermal delivery with chemical or physical approaches.

In some embodiments, the effective amount of the peptide is delivered orally for prevention and/or mitigation of e.g., chemotherapy and/or radiation-induced gastrointestinal injury.

### Methods for Treating Hair Loss in a Subject

The methods disclosed herein may be performed in order to treat hair loss, e.g., by promoting hair follicle neogenesis The disclosed methods typically comprise administering a peptide that is derived by treating the ECM with a collagenase, such as a bacterial collagenase, which releases peptides for use in the methods for treating hair loss. Hair follicle neogenesis requires trichogenic dermal papillar cells. The disclosed methods may be performed to induce trichogenic dermal papillar cells to form hair follicles in a subject in need thereof.

One aspect of the present disclosure provides a method for treating a subject having hair-loss, or a subject at risk for hair loss or progression of hair loss, or a subject with impaired hair growth function or loss of hair or other skin adnexal structures, the method comprising administering to the subject an effective amount of a peptide comprising the amino acid sequence of any of SEQ ID NOs: 1-19 or any combination thereof, for treating the hair-loss, or stimulating the formation, preservation, or function of hair follicles or other skin adnexa. In some embodiments, the peptide comprises the amino acid sequence of SEQ ID NO:6.

In the disclosed methods for treating hair loss, the peptide is combined with a carrier or excipient or transdermal delivery system. In some embodiments, the peptide is combined with follicular units or a substrate prior to administering the peptide to the subject, optionally via transplantation. Follicular units are complete anatomic and physiologic structures and comprise a small group of hair follicles and nerves blood vessels and erector pilorum muscles.

In the disclosed methods for treating hair loss, the peptide may be administered by any suitable route of administration. Suitable routes of administration may include oral administration, topical administration, subdermal administration, and/or subcutaneous administration. In some embodiments, the peptide is administered locally at the site of hair loss. In some embodiments, the peptide is administered ex vivo to cells, a substrate, or engineered skin or tissue prior to administration to the patient.

In some embodiments of the methods disclosed herein, an effective amount of the peptide is delivered at the site of hair loss for promoting neogenesis of hair follicles. In some embodiments, the subject is administered an effective amount of the peptide to achieve a concentration of at least about 1, 10, or 100 μM of peptide at the site of administration. In some embodiments, the subject is administered a therapeutically effective amount of a peptide with the amino acid sequence of SEQ ID NO: 6.

As used herein the term “effective amount” refers to the amount or dose of the compound that provides the desired effect. In some embodiments, the effective amount is the amount or dose of the compound, upon single or multiple dose administration to the subject, which provides the desired effect in the subject under diagnosis or treatment. Suitably the desired effect may be treating a subject having hair-loss.

An effective amount can be readily determined by those of skill in the art, including an attending physician and/or diagnostician, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. In some embodiments, the subject is responsive to therapy with the peptides disclosed herein, which, in some embodiments includes use in combination with one or more additional therapeutic agents. The term “treat” further includes the reduction in one or more symptom associated with hair-loss, for example, increase in hair growth, length, density or stimulating the formation, preservation, or function of hair follicles or other skin adnexa.

In the disclosed methods for treating hair loss, the peptide may be administered at any suitable frequency. In some embodiments, the subject may be administered the peptide daily or more frequently or at some other interval, such as every other day, every third day. In some embodiments, the subject may be administered the peptide for 1, 2, 3, 4, 5, 6 days, 1, 2, 3, 4 weeks or longer.

In the disclosed methods for treating hair loss, the peptide may be administered alone or as part of a combination therapy or complimentary therapy. In some embodiments the subject is administered an effective amount of the peptide and other therapies to treat hair loss. Other therapies to treat hair loss include, but are not limited to Minoxidil, Finasteride, Baricitinib, spironolactone, dutasteride, corticosteroids, platelet-rich plasma, hair transplant surgery (e.g. follicular unit transplantation, follicular unit excision, robotic follicular unit excision techniques), follicular cell implantation, laser therapy, microneedling, or the Follica device.

Suitable subjects for the disclosed methods of treating hair loss may include subjects which have lost hair follicles. Subjects for the disclosed methods of treating hair loss may have lost hair follicles due to age, for example, where the subject is at least 20, 30, 40, 50, or 60 years of age.

Suitable subjects for the disclosed methods of treating hair loss may include subjects which have lost hair follicles due to the effects of testosterone. Subjects for the disclosed methods of treating hair loss may have hair loss that is attributed to androgenetic alopecia.

Suitable subjects for the disclosed methods of treating hair loss may include subjects which have lost hair follicles due to other conditions. Subjects for the disclosed methods of treating hair loss may have hair loss that is attributed to alopecia areata, anagen effluvium, telogen effluvium, tinea capitis, or traction alopecia.

Suitable subjects for the disclosed methods of treating hair loss may include subjects which have lost hair follicles due to trauma, and optionally physical trauma, thermal trauma, chemical trauma, or radiological trauma or other forms of trauma. In some embodiments the trauma results in full or partial skin loss and/or reduction in skin thickness.

Suitable subjects for the disclosed methods of treating hair loss may include subjects which have lost hair follicles due to a burn wound. Subjects for the disclosed methods of treating hair loss may have hair loss that is attributed to a burn wound that has been caused by heat, radiation therapy, or a combination thereof.

Suitable subjects for the disclosed methods of treating hair loss may include subjects which have lost hair follicles due a treatment or therapy for another condition or disease. In some embodiments, the subject has hair-loss attributed to a treatment that results in the death of hair follicles.

Suitable subjects for the disclosed methods of treating hair loss may include subjects which have lost hair follicles due to radiation therapy. Subjects for the disclosed methods of treating hair loss may have cancer and may have hair loss attributed to radiation therapy or chemotherapy treatment.

Suitable subjects for the disclosed methods of treating hair loss may include subjects who want to prevent or mitigate the loss of hair due to age, the effect of hormones including testosterone, disease or other condition, trauma, burn, therapy, or radiation.

In some embodiments, efficacy is determined by comparison with one or more untreated control subject, wherein the control subject has experienced the same or similar condition and concomitant hair loss, and has not been administered the peptides of the present disclosure. Regarding therapeutic efficacy, in some embodiments, a mitigating effect is noted (e.g., less hair loss, or improved hair growth or hair follicle formation) within 1 week, 2 weeks, 3 weeks, 1 month, 2 months, 3 months, or within about 4 months of the first therapeutic dose of the peptide. In some embodiment, the peptide may exhibit sustained efficacy days, weeks or longer after administration.

In the disclosed methods for mitigating hair loss, the peptide may be administered at any suitable frequency. In some embodiments, the subject may be administered the peptide daily or more frequently. In some embodiments, the subject may be administered the peptide daily for 1, 2, 3, 4, 5, 6 days, 1, 2, 3, 4 weeks or longer. In some embodiments, the administration is daily, weekly, bi-weekly, monthly, or any combination thereof.

In the disclosed methods, subjects may be treated with the peptide before, and/or during, and/or after a detected disease, treatment, or other situation in which hair loss is noted and/or is an expected outcome, side effect, or symptom.

Methods for Prevention and/or Mitigating the Negative Effects of Disease, Therapy, or Radiation Exposure in a Subject

The methods disclosed herein may be performed for preventing and/or mitigating the effects of disease, chemotherapy, radiation therapy, or other radiation exposure event (e.g., accidental radiation exposure or exposure due to a military/combat event, or other radiation releasing event). In particular, the methods relate to mitigating hair loss associated with the aforementioned diseases, treatments, or exposures, and/or increasing hair growth after or during such disease, treatment or exposure. The methods typically comprise administering to the subject an effective amount of a peptide comprising the amino acid sequence of any of SEQ ID NOs: 1-19 or any combination thereof, e.g., for mitigating the effects of radiation therapy or other radiation exposure. In some embodiments the methods comprise administering an effective amount of a peptide comprising the amino acid sequence of SEQ ID NO: 6 and/or SEQ ID NO: 18.

In some embodiments, the peptide is administered after radiation exposure, a therapy, or disease diagnosis. In some embodiments, the peptide is administered prior to e.g., therapy, radiation exposure, or disease diagnosis. In some embodiment, the peptide exhibits sustained efficacy days, weeks, or longer after administration In some embodiments, the peptide is administered at the time of therapy, radiation exposure, or disease diagnosis. In some embodiment, the peptide exhibits sustained efficacy days, weeks, or longer after administration. By way of example and not by way of limitation, in some embodiments, the peptide is administered after therapy, radiation exposure, or disease diagnosis, e.g., about 1 hour, 2 hours, 6 hours, 12 hours, 18 hours, 24 hours, 28 hours, 32 hours, 36 hours, 40 hours, 48 hours or more after disease diagnosis, treatment, or exposure, and exhibits a therapeutic effect days, weeks or longer after a first administration (e.g., mitigation or prevention of one or more symptoms, such as hair loss). By way of example and not by way of limitation, in some embodiments, the peptide is administered prior to disease diagnosis, therapy, or exposure, e.g., about 1 hour, 2 hours, 6 hours, 12 hours, 18 hours, 24 hours, 28 hours, 32 hours, 36 hours, 40 hours, 48 hours or more before disease diagnosis, therapy, or exposure, and exhibits a therapeutic effect days, weeks, or longer (e.g., mitigation or prevention of one or more symptoms such as hair loss) after the radiation exposure. In some embodiments, the peptide is administered before and after radiation exposure and may have sustained efficacy days, weeks or longer after administration. In some embodiments, the peptide is administered before, and/or during, and/or after therapy, radiation exposure, or disease diagnosis and may have sustained efficacy days, weeks or longer after administration. Mitigation of hair loss includes improved hair growth.

In some embodiments, efficacy is determined by comparison with one or more untreated control subject, wherein the control subject has experienced the same or similar disease, therapy, or radiation exposure and has not been administered the peptides of the present disclosure. Regarding therapeutic efficacy, in some embodiments, a mitigating effect is noted (e.g., less hair loss, or improved hair growth or hair follicle formation) within 1 week, 2, weeks, 3 weeks, 1 month, 2 months, 3 months, or within about 4 months of the first therapeutic dose of the peptide.

In some embodiments of the disclosed methods, the subject has cancer and the subject is administered radiation therapy in order to treat the cancer. In some embodiments, the subject has cancer and is treated with chemotherapy to treat the cancer. Radiation therapy can result in the loss of hair at the site of treatment. Chemotherapy treatment is systemic and can damage hair follicles and result in hair loss all over the body. Subjects may be treated with radiation therapy, chemotherapy or a combination of the two. Methods disclosed herein may be suitable for mitigating or treating cancer treatment associated hair loss. In some embodiments, the peptides may be suitable for use in the disclosed methods in subjects exposed to radiation therapy and/or chemotherapy.

In other embodiments, the subject has a disease other than cancer, such as Dupuytren's Disease or Ledderhose disease, and the subject is administered radiation therapy in order to treat the other disease. In further embodiments, the subject has a benign tumor, a thyroid disease, or a blood disorder, and the subject is administered radiation therapy to treat the benign tumor, the thyroid disease, or the blood disorder.

The subject may be administered radiation therapy and the subject subsequently may be administered an effective amount of a peptide after radiation exposure for mitigating the effects of the radiation therapy. In some embodiments, the subject may be administered an effective amount of a peptide prophylactically to counteract the effects of the radiation therapy prior the subject being administered the radiation therapy.

In the methods for prevention of or mitigating the subsequent negative effects of radiation therapy, the subject may be administered the peptide by any suitable route prior to receiving radiation treatment(s). Suitable routes of administration may include oral, topical administration, subdermal administration, and/or subcutaneous administration. In some embodiments, the peptide is administered locally at the site where radiation therapy is administered.

In the methods for mitigating the negative effects of radiation therapy, the subject may be administered the peptide by any suitable route after radiation therapy. Suitable routes of administration may include topical administration, subdermal administration, and/or subcutaneous administration. In some embodiments, the peptide is administered locally at the site where radiation therapy is administered. Examples of topical administration include, but are not limited to gels, ointments, creams, and suspensions, dressings, bioerodible patches, follicular units, ex-vivo cells, scaffolds, skin equivalents or transdermal delivery with chemical or physical approaches.

In the methods for mitigating the negative effects of radiation therapy, the subject may be administered an effective amount of the peptide for treating dermatitis, including erythema, scale, and ulceration, at the site at which radiation therapy is administered. In some embodiments, the subject may be administered an effective amount of the peptide for mitigating stem cell senescence in the skin at the site at which radiation therapy is administered.

In the methods for mitigating the negative effects of radiation therapy, the subject may be administered a dose of radiation which results in the loss or death of hair follicles at the site at which the dose of radiation is administered. The subject may have been accidentally exposed to radiation or may have been exposed due to military conflict. The subject may be administered an effective amount of the peptide before and/or after the subject is administered radiation therapy, where the effective amount of the peptide is effective for promoting neogenesis of hair follicles at the site at which the radiation therapy is administered. In some embodiments, the peptide is administered after radiation exposure. In some embodiment, the peptide may be administered before, and/or during, and/or after radiation exposure and may have sustained efficacy days, weeks or longer after administration.

Preferably in the disclosed methods an effective amount of the peptide is delivered at the site where radiation therapy is administered for mitigating negative effects of the radiation therapy. In some embodiments, the subject is administered an effective amount of the peptide to achieve a concentration of at least about 1, 10, or 100 μM at the site of administration.

In the disclosed methods for mitigating the negative effects of disease, therapy, or radiation exposure, the peptide may be administered at any suitable frequency. In some embodiments, the subject may be administered the peptide daily or more frequently. In some embodiments, the subject may be administered the peptide daily for 1, 2, 3, 4, 5, 6 days, 1, 2, 3, 4 weeks or longer. In some embodiments, the administration is daily, weekly, bi-weekly, monthly, or any combination thereof.

In some embodiments of the disclosed methods, the subject has been exposed to non-therapeutic radiation. Non-therapeutic radiation may comprise any radiation exposure that is not for therapeutic purposes. Non-therapeutic radiation includes ionizing radiation or non-ionizing radiation. Examples of ionizing radiation include, but are not limited to nuclear radiation, X-rays and gamma rays from radioactive elements. Examples of non-ionizing radiation include but are not limited to infrared radiation and UV radiation (including solar, and artificial UV such as tanning beds and medical devices). In some embodiments, the subject may have been exposed to radiation accidentally or due to a military conflict involving the deployment of radiation. A subject may be administered a therapeutic amount of a peptide of the present disclosure before, during and/or after such an exposure.

In the disclosed methods for mitigating the negative effects of hair loss generally, or hair loss due to disease, therapy, or radiation exposure, the peptide may be administered alone or in combination with other therapies or used in conjunction with other therapies. Other therapies include, but are not limited to the RECELL® system, allogeneic cellularized scaffold products including StrataGraft®, skin scaffolds including naturally derived or bioengineered scaffolds, bioengineered skin substitutes or skin constructs. The disclosed peptides may also be administered with skin grafts including split thickness grafts, full thickness grafts, and composite grafts. The disclosed peptides may also be encapsulated for administration, or administered via bio-erodible microneedle or microneedle array patches.

### Additional Definitions

The technology provided herein is not limited to the particular methodologies, protocols, constructs, formulae and reagents described but further include those known to the skilled artisan. It is also to be understood that the terminology used herein is for the purpose of describing particular embodiments only and is not intended to limit the scope of the technology provided herein.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising.” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus>10% of the particular term.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

As used herein, the terms “subject” and “patient” are used interchangeably, and refer to the individual of interest (e.g., the individual, such as a mammal, e.g., a human), to be treated with the peptides of the present disclosure, or that are serving as “no-treatment” controls, for example.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

Unless defined otherwise, all technical and scientific terms used herein have the same meaning as commonly understood to one of ordinary skill in the art to which technology provided herein belongs. Any methods, materials, and kits similar or equivalent to those described herein can be used in the practice or testing of the technology provided herein.

### EXEMPLARY EMBODIMENTS

Embodiment 1. A method for treating a subject having hair-loss, or a subject at risk for hair loss or progression of hair loss, or a subject with impaired function or loss of hair or other skin adnexal structures, the method comprising administering to the subject an effective amount of a peptide comprising the amino acid sequence of any of SEQ ID NOs: 1-19 for treating the hair-loss, or stimulating the formation, preservation, or function of hair follicles or other skin adnexa.

Embodiment 2. The method of embodiment 1, wherein the peptide consists of the amino acid sequence of any of SEQ ID NOs: 1-19.

Embodiment 3. The method of any of the previous embodiments, wherein the peptide has a length of 8, 10, 20, 30, 40, 50, 60, 70, 80, 90, or 100 amino acids, or a length within a range bounded by any of 8, 10, 20, 30, 40, 50, 60, 70, 80, 90, or 100 amino acids (e.g., a length of 8-100 amino acids).

Embodiment 4. The method of any of the previous embodiments, wherein the peptide comprises no more than 50, 45, 40, 35, 30, 25, or 20 contiguous amino acid residues of the protein from which the peptide is derived.

Embodiment 5. The method of any of the previous embodiments, wherein the peptide consists of a combinatorial peptide comprising one or more SEQ ID NOs: 1-19 fused to a second peptide or polypeptide.

Embodiment 6. The method of any of the previous embodiments, wherein the peptide comprises the amino acid sequence of SEQ ID NO:6.

Embodiment 7. The method of any of the previous embodiments, where in the peptide is combined with a carrier or excipient or transdermal delivery system, optionally wherein the peptide is combined with follicular units or a substrate prior to administering the peptide to the subject, optionally via transplantation.

Embodiment 8. The method of any of the previous embodiments, wherein the peptide is administered locally to the treatment area, optionally wherein the peptide is administered ex vivo to cells, a substrate, or engineered skin prior to administration to the patient.

Embodiment 9. The method of any of the previous embodiments, wherein the effective amount of the peptide is effective for promoting neogenesis or preservation of hair follicles or improvement in hair follicle function.

Embodiment 10. The method of any of the previous embodiments, wherein the effective amount of the peptide is administered topically.

Embodiment 11. The method of any of the previous embodiments, wherein the effective amount of the peptide is administered subcutaneously.

Embodiment 12. The method of any of the previous embodiments, wherein the effective amount of the peptide is administered intradermally.

Embodiment 13. The method of any of the previous embodiments, wherein the subject is administered an effective amount of the peptide to achieve a concentration of at least about. 1, 1, 10, 100 or 1000 μM at the site of administration.

Embodiment 14. The method of any of the previous embodiments, wherein the subject is administered the effective amount daily.

Embodiment 15. The method of any of the previous embodiments, wherein the subject has hair-loss attributed to the loss of hair follicles.

Embodiment 16. The method of any of the previous embodiments, wherein the subject is at least 20, 30, 40, 50, or 60 years of age.

Embodiment 17. The method of any of the previous embodiments, wherein the subject has hair-loss attributed to androgenetic alopecia.

Embodiment 18. The method of any of the previous embodiments, wherein the subject has hair-loss due to trauma, and optionally physical trauma, thermal trauma, chemical trauma, or radiological trauma or other forms of trauma.

Embodiment 19. The method of any of the previous embodiments, wherein the trauma results in full or partial skin loss and/or reduction in skin thickness.

Embodiment 20. The method of any of the previous embodiments, wherein the subject has a scarring form of alopecia optionally selected from central centrifugal cicatricial alopecia, lichen planopilaris, acne keloidalis nuchae, dissecting cellulitis, traction alopecia, and pseudopelade of Brocq.

Embodiment 21. The method of any of the previous embodiments, wherein the subject has a non-scarring form of alopecia, optionally selected from alopecia areata, anagen effluvium, and telogen effluvium.

Embodiment 22. The method of any of the previous embodiments, wherein the subject has hair-loss attributed to a burn wound.

Embodiment 23. The method of any of the previous embodiments, wherein the subject has hair-loss attributed to a treatment that results in the death of hair follicles.

Embodiment 24. The method of any of the previous embodiments, wherein the subject has hair-loss attributed to radiation treatment or chemotherapy treatment.

Embodiment 25. The method of any of the previous embodiments, wherein the subject has cancer and has undergone radiation or chemotherapy treatment.

Embodiment 26. A method for mitigating the effects of radiation therapy in a subject undergoing radiation therapy, the method comprising administering to the subject an effective amount of an effective amount of a peptide comprising the amino acid sequence of any of SEQ ID NOs: 1-19 for mitigating the effects of radiation therapy.

Embodiment 27. The method of embodiment 26, wherein the peptide consists of the amino acid sequence of any of SEQ ID NOs: 1-19.

Embodiment 28. The method of any of embodiments 26-27, wherein the peptide has a length of 8, 10, 20, 30, 40, 50, 60, 70, 80, 90, or 100 amino acids, or a length within a range bounded by any of 8, 10, 20, 30, 40, 50, 60, 70, 80, 90, or 100 amino acids (e.g., a length of 8-100 amino acids).

Embodiment 29. The method of any of embodiments 26-28, wherein the peptide comprises no more than 50, 45, 40, 35, 30, 25, or 20 contiguous amino acid residues of the protein from which the peptide is derived.

Embodiment 30. The method of any of embodiments 26-29, wherein the peptide consists of a combinatorial peptide comprising one or more SEQ ID NOs: 1-19 fused to a second peptide or polypeptide.

Embodiment 31. The method of any of embodiments 26-30, wherein the peptide comprises the amino acid sequence of SEQ ID NO:6.

Embodiment 32. The method of any of embodiments 26-31, where in the peptide is combined with a carrier or excipient or transdermal delivery system.

Embodiment 33. The method of any of embodiments 26-32, where in the peptide is administered locally at the site where radiation therapy is administered.

Embodiment 34. The method of any of embodiments 26-33, wherein the effective amount of the peptide is administered topically.

Embodiment 35. The method of any of embodiments 26-34, wherein the effective amount of the peptide is administered subcutaneously.

Embodiment 36. The method of any of embodiments 26-35, wherein the effective amount of the peptide is administered intradermally.

Embodiment 37. The method of any of embodiments 26-36, wherein the effective amount of the peptide is effective for treating dermatitis.

Embodiment 38. The method of any of embodiments 26-37, wherein the effective amount of the peptide is effective for mitigating stem cell senescence in the skin.

Embodiment 39. The method of any of embodiments 26-38, wherein the effective amount of the peptide is effective for promoting neogenesis or preservation of hair follicles or hair follicle function.

Embodiment 40. The method of any of embodiments 26-39, wherein the subject is administered the effective amount daily.

Embodiment 41. The method of any of embodiments 26-40, wherein the subject is administered a dose of radiation that results in the death of hair follicles.

Embodiment 42. The method of any of embodiments 26-41, wherein the subject has cancer and the subject is undergoing radiation therapy to treat the cancer.

Embodiment 43. The method of any of embodiments 26-42, wherein the subject has cancer, the subject is undergoing radiation therapy to treat the cancer, or chemotherapy to treat cancer, and the effective amount of the peptide is administered at the site where radiation therapy is administered.

### EXAMPLES

The following examples are included merely for purposes of illustration of certain embodiments and embodiments of the present disclosure and are not intended to limit the invention.

### Example 1: TSN Peptides Enhanced Hair Follicle Neogenesis

The active pharmaceutical ingredient (API) enzyme(s) present within SANTYL® collagenase ointment were used to produce peptides from well-defined, bio-synthesized extracellular matrices derived from living, human epidermal and dermal cell cultures in vitro⁷. Fifteen collagenous and collagen-associated ECM peptides, of 8-21 amino acids in length, have now identified, sequenced and synthesized (TSN peptides)⁷. Mass spectrometric identification of releases reveals a cell type-specific array of peptides including key peptides derived from the extracellular matrix-bound and collagen-associated macromolecules (i) thrombospondin, (ii) multimerin and (iii) fibronectin, which are collagenase-sensitive and released from endothelial ECM. In addition, collagenase yields peptides from (i) TGF-β-induced protein and (ii) collagen VI, which are derived from ECM synthesized and organized by human dermal fibroblasts. Peptides have been synthesized from these original structures bearing hydrophobicity and hydrophilicity profiles, which insures that each peptide is soluble in water⁷.

In addition to these peptides, which were produced by collagenase cleavage of synthesized endothelial or fibroblastic matrices, four peptides were “re-engineered”. These re-engineered peptides are not naturally produced via collagenase-digestion; the collagenase-produced “parent” peptide(s) were re-engineered to optimize and maximize wound healing potential⁷. These include peptides TSN9, TSN10, TSN18, TSN19, which possess amino acid sequence identity to, at least, two distinct collagenase-liberated peptides identified by mass spectrometry (Table 1). The peptides were synthesized at Tufts University Core Facility and their biological activity evaluated in a series of tests, including (i) ability to stimulate cell-specific proliferation, (ii) activation of angiogenesis and (iii) wound healing, in vitro and in vivo. TSN Peptides 1-9 are derived from Human Dermal Endothelial Cell Extracellular Matrix. TSN Peptides 10-19 are derived from Human Dermal Fibroblast Extracellular Matrix⁸.

TABLE 1
Isolated TSN Protease-Resistant Therapeutic Peptides

| Peptide | | | |
|---|---|---|---|
| Name | SEQ ID NO: | Sequence | Origin |
| TSN1 | 1 | NFQGVQNRFVFGTP | Thrombospondin-1 Laminin |
| | | | G-like domain, heparin- |
| | | | binding N-terminal domain |
| TSN2 | 2 | MENAELDVPIQSVFTR | Thrombospondin-1 N- |
| | | | terminal domain, heparin- |
| | | | binding N-terminal domain |
| TSN3 | 3 | NTDNIYPESSC | Multimerin EGF-like |
| | | | domain |
| TSN4 | 4 | PYLGYVFK | Multimerin Clq domain |
| TSN5 | 5 | MQTVAQLFKTVSSLSLST | Multimerin-1 Coiled coil |
| | | | domain |
| TSN6 | 6 | HSPDIQLQKGLTFEPIQIK | Multimerin-1 Coiled coil |
| | | | domain |
| TSN7 | 7 | STITQPYKTLNNARSP | Fibronectin Heparin- binding |
| | | | domain 14, heparin-binding |
| | | | region 2 |
| TSN8 | 8 | RPGPSPEGTGQSYNY | Fibronectin Fibrin- binding 2 |
| | | | domain 12, heparin-binding |
| | | | region 2 |
| TSN9 | 9 | MENAELDPPYLGYVSK | Combination of |
| | | | Thrombospondin and |
| | | | multimerin peptides |
| TSN10 | 10 | TGQSYNQYSQRPYLGVYVFK | Combination of |
| | | | Thrombospondin and |
| | | | multimerin peptides |
| TSN11 | 11 | LYGQTPLETL | TGF-ß-induced protein: |
| | | | Fas1/3 domain, Fasciclin |
| | | | domain 3 |
| TSN12 | 12 | ELADSPALEIG | TGF-beta-induced protein: |
| | | | N-terminal domain |
| TSN13 | 13 | LYGQTPLETLELADSPALEIG | Combination of TSN11 and |
| | | | TSN12 |
| TSN14 | 14 | VSGNTVEYALPTLE | Tenascin C Fibronectin Type |
| | | | III domain 14 |
| TSN15 | 15 | LDSPTAPTVQSTALTWRP | Tenascin C Fibronectin III |
| | | | domain 15 |
| TSN16 | 16 | LDGSAPGPLYTGSALDF | Collagen (VI) Alpha-3 |
| | | | VWFA domain 3 |
| TSN17 | 17 | GSEGVRSGRSG | Collagen (VI) Alpha-3 |
| | | | VWFA domain 6 |
| TSN18 | 18 | QPQPLPSPGVGGKN | Combination of non-helical |
| | | | Collagen (VI) Alpha-3 chain |
| | | | regions |
| TSN19 | 19 | KYTLNPVIDAS | Combination of fibronectin |
| | | | Type-III 14 domain of |
| | | | fibronectin |

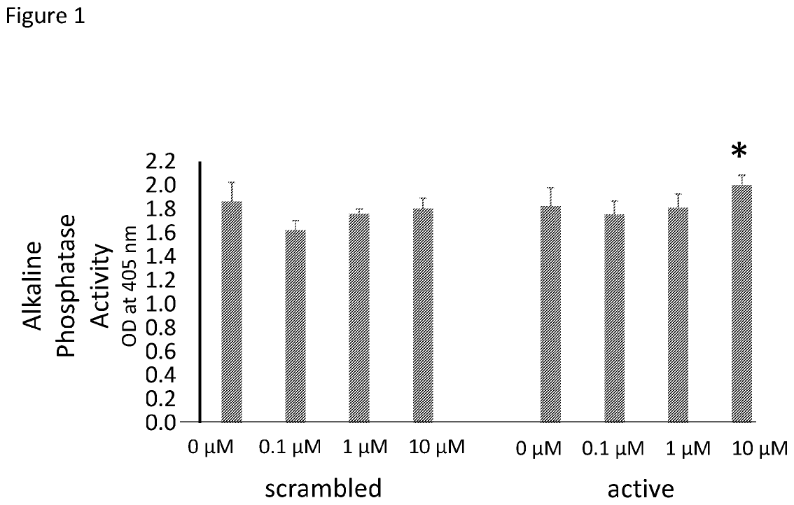

Dermal papilla cells regulate hair follicle development and growth, unfortunately, maintaining dermal papilla cells inductive capacity is difficult in culture, as evident by their gradual loss of alkaline phosphatase activity in culture³. To test if TSN peptides can increase alkaline phosphatase activity keratinocytes and dermal papilla cells were plated at 4000 and 2000 cells/well respectively in a 96 well plate with six replicates and cultured overnight. The media was changed and appropriate treatment-vehicle, scrambled peptide or active peptide—was added to the well. The media were changed with fresh media containing peptides 2 days after initial treatment, and the total culture period was 4 days, following which alkaline phosphatase activity was measured (FIG. 1). 10 μM of active TSN peptides increased alkaline phosphatase activity compared to scrambled peptides.

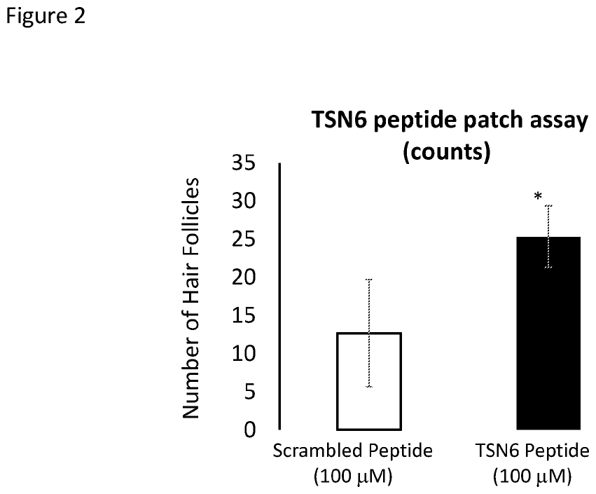

Next, we used a patch assay to test is TSN peptides can induce hair follicle neogenesis in vivo. Epidermal aggregates were resuspended in DMEM/F12 at 750,000 aggregates/750 μL (1000 aggregates/μL) and spheroids were resuspended in DMEM/F12 at 1400 spheroids/140 μL (10 spheroids/μL). 10 million dermal cells were resuspended in 400 μL DMEM/F12 (25,000 cell/μL or 1.25E6 per 50 μL). The appropriate tubes for positive control (30 μL (30,000) aggregates and 120 μL mouse dermal cells (3 million) and 30 μL DMEM/F12), no DP cells control (30 μL (30,000) aggregates and 150 μL DMEM/F12), scrambled peptide control (175 μL epidermal aggregates (175,000 aggregates), 70 μL spheroids (700 spheroids) and 6.25 μL (4 mg/mL) scrambled peptide), active peptide ((175 μL epidermal aggregates (175,000 aggregates), 70 μL spheroids (700 spheroids) and 6.25 μL (4 mg/mL) active peptide) were prepared. The solutions were kept on ice until injection into mature 6-10 week old nu/nu mouse. Up to 8 injections may be placed in each mouse. The cells were injected into the deep dermis slowly without injecting the air bubble or penetrating the whole dermis so cells can remain in a confined space in the dermis. The implanted cells were allowed to develop for 2-4 weeks. Using a dissecting microscope the number of hair follicles were counted for each implantation from the mucosal side of the skin (FIG. 2). TSN peptides induced a two-fold increase in hair follicle formation.

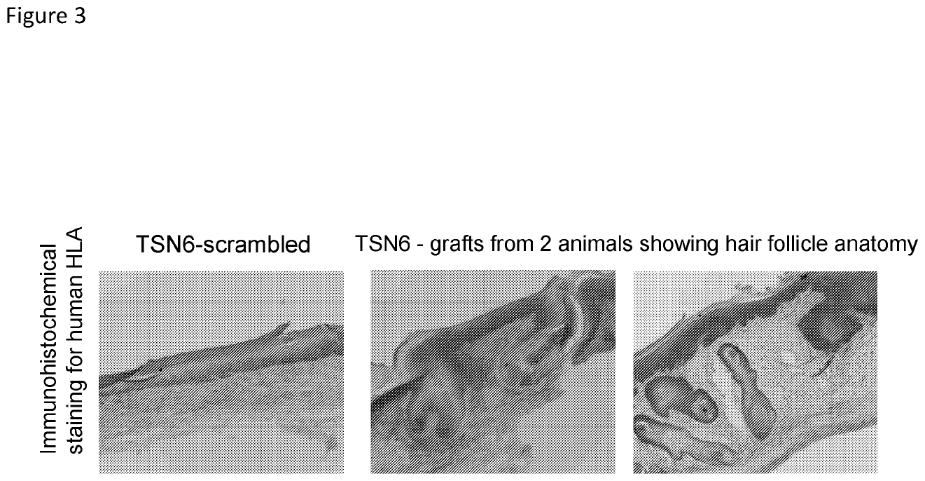

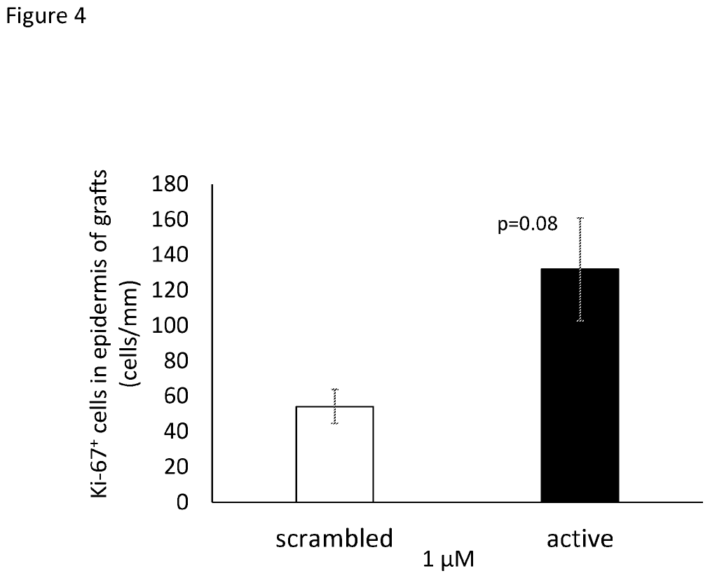

We next asked if TNS peptides could increase human hair follicle formation in grafted dermal-epidermal composite cultures. Human dermal papilla cells isolated from temporal scalp dermis and propagated in vitro. Normal foreskin keratinocytes (NFK) were isolated from neonatal foreskins (pooled from 5-7 donors) and were cultured in serum-free medium supplemented with EGF and bovine pituitary extract. Dermal epidermal composites (DECs) were prepared with human dermal papilla cells and NFK and were grafted onto nude mice. Imaging was done at 4 weeks, 6 weeks, 8 weeks, 10 weeks and 12 weeks and staining for hHLA was performed to evaluate hair follicle anatomy (FIG. 3). TSN peptides increased hair follicle generation (FIG. 3) as well as increased epidermal proliferation in vivo (FIG. 4).

### Methods

Cell viability assay: Keratinocytes and dermal papilla cells were plated at 4000 and 2000 cells/well respectively in a 96 well plate with six replicates and cultured overnight. The media was changed and appropriate treatment-vehicle, scrambled peptide or active peptide—was added to the well. The media were changed with fresh media containing peptides 2 days after initial treatment, and the total culture period was 4 days. The cell viability was then measured using CellTiter 960 Non-Radioactive Cell Proliferation Assay (MTT) (Promega Corporation). Briefly, 15 μL of dye solution was added to each well and incubated for 4 h at 37° C. and 5% CO₂. Next 100 μL of stop solution was added to the mixture, incubated for an hour and absorbance was recorded at 570 nm using a plate reader.

Alkaline Phosphatase activity: Keratinocytes and dermal papilla cells were plated at 4000 and 2000 cells/well respectively in a 96 well plate with six replicates and cultured overnight. The media was changed and appropriate treatment-vehicle, scrambled peptide or active peptide—was added to the well. The media were changed with fresh media containing peptides 2 days after initial treatment, and the total culture period was 4 days. Alkaline phosphatase activity was measured according to manufacturer's instructions using Alkaline Phosphatase Yellow (pNPP) Liquid Substrate System for ELISA (Sigma Aldrich).

Preparation of spheroids: Dermal Papilla (DP) cells were trypsinized, neutralized and counted. The cells were centrifuged and resuspended as 6.5×10⁶ cells in 30 mL DP cell media. 0.9 mL of collagen (3.5 mg/mL stock) was added to form is 4285 cells/20 μL in DP media with 0.1 mg/mL collagen. 20 μL droplets of cells (4000 cells/droplet) were pipetted on the bottom of 10 cm tissue culture dish lid. About 8-10 mL PBS was added to bottom of dish. The lid was placed on the dish and incubated at 37° C. for 48 hours.

Preparation of epidermal Cell aggregates: From 12 neonatal foreskins, the epidermis was separated from the dermis using dispase incubation overnight. The epidermis was then minced to form a fine paste and then incubated in trypsin (0.025% diluted in DPBS at 37° C.) with periodic swirling for 20 min. The trypsin was then neutralized with trypsin neutralizing solution and the solution was gravity filtered. The filtrate was washed with 10 mL DMEM three times. This was then centrifuged at 800 rpm for 5 minutes. The aggregates pelleted were resuspended in 16 mL of 4.5% ficol solution and 8 mL of the 4.5% ficol/aggregate suspension was layered carefully onto 9% ficol solution. The tube was centrifuged at 80 0 rpm for 5 min. The supernatant was aspirated and the pellet was washed twice with 15 mL DMEM. The final pellet was resuspended in 6 mL DMEM/F12 (for 12 skins) and the aggregates were counted using hemocytometer.

Patch assay: Epidermal aggregates were resuspended in DMEM/F12 at 750,000 aggregates/750 μL (1000 aggregates/μL) and spheroids were resuspended in DMEM/F12 at 1400 spheroids/140 μL (10 spheroids/μL). 10 million dermal cells were resuspended in 400 μL DMEM/F12 (25,000 cell/μL or 1.25E6 per 50 μL). The appropriate tubes for positive control (30 μL (30,000) aggregates and 120 μL mouse dermal cells (3 million) and 30 μL DMEM/F12), no DP cells control (30 μL (30,000) aggregates and 150 μL DMEM/F12), scrambled peptide control (175 μL epidermal aggregates (175,000 aggregates), 70 μL spheroids (700 spheroids) and 6.25 μL (4 mg/mL) scrambled peptide), active peptide ((175 μL epidermal aggregates (175,000 aggregates), 70 μL spheroids (700 spheroids) and 6.25 μL (4 mg/mL) active peptide) were prepared. The solutions were kept on ice till injection. Mature 6-10 week old nu/nu mouse were anesthetized and the truncal skin surface was cleansed with 70% alcohol wipes. From the cell mixture, the excess medium was removed the cell pellet was resuspended for injection in 60 to 80 μL of DMEM/F12 per injection (˜150 μL for two injections). A syringe with a 25 gauge needle was loaded allowing an air bubble behind the sample and the base of the plunger but avoid introducing air bubbles into the cell suspension. Up to 8 injections may be placed in each mouse. The cells were injected into the deep dermis slowly without injecting the air bubble or penetrating the whole dermis so cells can remain in a confined space in the dermis. The implanted cells were allowed to develop for 2-4 weeks as mouse dermal and mouse epidermal cells will form hair follicles within 12 days of delivery. In contrast human epidermal cells and mouse dermal cells need ˜17 days to form new hair follicles and human dermal/mouse epidermal cells need 21 to 26 days. The site of implantation was tattooed by piercing the skin peripheral side of the injection site once with needle and labeled. At the end of the incubation period, mice were sacrificed by CO₂ narcosis. Full-thickness skin was collected and fixed in 10% formalin. Using a dissecting microscope the number of hair follicles were counted for each implantation from the mucosal side of the skin.

Dermal-epidermal composites and grafting: Human dermal papilla cells isolated from temporal scalp dermis (Promocell, Heidelberg, Germany) from a female donor (HDP47) were propagated in vitro according to manufacturer's recommendations. Normal foreskin keratinocytes (NFK) were isolated from neonatal foreskins (pooled from 5-7 donors) and were cultured in serum-free medium (Keratinocyte-SFM, Invitrogen), supplemented with EGF and bovine pituitary extract according to manufacturer's recommendation. Dermal epidermal composites (DECs) were prepared with human dermal papilla cells and NFK and were grafted onto nude mice. Briefly, dermal equivalents with dissociated human DP cells were (Passage 8, 500,000 cells/composite) suspended in 1 mg/ml of rat tail collagen type 1 (BD Biosciences, MA) in 24-mm insert collagen coated permeable supports (Costar, #3492). These dermal constructs were incubated in epidermalization medium for 3 days before adding 10⁶ human NFK with accompanying melanocytes (primary (P0). passage 1 (P1) and passage 3 (P3), n=8/group) on top. Melanocytes persist in culture for seven passages when primary keratinocytes are passaged without freezing. These DECs were cultured submerged in epidermalization medium and subsequently brought to the air-liquid interface using cornification medium and were grafted to 6-8 weeks old NIH(S)-nu/nu female mice. The surgical site was covered with petrolatum gauze and secured with bandages. The bandages were changed at 2 weeks and removed after 4 weeks. Imaging was done at 4 weeks, 6 weeks, 8 weeks, 10 weeks, and 12 weeks. All animal experiments were performed in accordance to our Institutional Animal Care and Use Committee guidelines.

Histology: H&E Staining: Unstained slides were deparaffinized in xylene, then hydrated through graded alcohols up to water. Then they were placed in Carazzi's hematoxylin, washed in tap water, and then placed in 95% ethanol. From there they were placed in eosin-phloxine solution, and then dehydrated through graded alcohols to xylene. After xylene, the stained slides were cover-slipped using Permount as the mounting media.

HLA staining: Frozen section slides were air dried for 20 min and then fixed in-20° C. cold acetone for 3 min. The slides were then rinsed in PBS twice for 5 min each. The sections were outlined and incubated in blocking solution (BTPBS) with 10% normal goat serum for 1 hour at RT. The excess blocking serum was wiped off. The slides were then incubated with 100 μL of diluted specific primary antibody (HLA) or negative control at 4° C. in humidified chamber overnight. The next day the slides were allowed to warm to room temperature. The slides were rinsed with PBS thrice for 5 min each and then incubated with ABC reagent at RT for 30 min. The slides were rinsed with PBS thrice for 5 min each. The slides were incubated in alkaline phosphatase substrate solution for 30 min at RT and then rinsed in tap water. The slides were then incubated with Hematoxylin for 5 min and rinsed in running water until rinse water is colorless. Finally, the slides were dehydrated using ethanol and xylene following 70% ethanol for 30 sec, then 95% ethanol for 30 sec, then 100% ethanol for 30 sec and then xylenes for 5 min. The slides were wiped to remove excess xylenes and Permanent Mounting Medium was added and then covered using a coverslip.

Image analysis: Both H&E and HLA slides were scanned using Zeiss Axioscan scanner and analyzed using Zen Lite (Zeiss) and ImageJ (NIH) software. The slides were scored by a blinded pathologist for hair follicle presence/absence, hair follicle density and total area of hair follicles.

### REFERENCES, EXAMPLE 1

1. Kurt S Stenn, George Cotsarelis Bioengineering the hair follicle: fringe benefits of stem cell technology. Current Opinion in Biotechnology. Volume 16, Issue 5. 2005, 493-497.
2. Marshall, Clement D et al. “Cutaneous Scarring: Basic Science, Current Treatments, and Future Directions.” Advances in wound care vol. 7, 2 (2018): 29-45. doi: 10.1089/wound.2016.0696.
3. Thangapazham R L, Klover P, Wang J A, Zheng Y, Devine A, Li S, Sperling L, Cotsarelis G, Darling T N. Dissociated human dermal papilla cells induce hair follicle neogenesis in grafted dermal-epidermal composites. J Invest Dermatol. 2014 February; 134(2): 538-540.
4. Iacovelli, Nicola Alessandro et al. “Topical treatment of radiation-induced dermatitis: current issues and potential solutions.” Drugs in context vol. 9 2020 Apr. 7. 12 Jun. 2020, doi: 10.7573/dic.2020-4-7.
5. https://my.clevelandclinic.org/health/diseases/21995-radiation-burns
6. Kawamura, M., Yoshimura, M., Asada, H. et al. A scoring system predicting acute radiation dermatitis in patients with head and neck cancer treated with intensity-modulated radiotherapy. Radiat Oncol 14, 14 (2019). https://doi.org/10.1186/s13014-019-1215-2.
7. Herman. I. (2019). Collagenase-derived peptides promote Tissue Regeneration and Wound Healing. (U.S. Pat. No. 10,485,846 B2). United States Patent Office.
8. Herman I M, Castellot J J Jr. Regulation of vascular smooth muscle cell growth by endothelial-synthesized extracellular matrices. Arteriosclerosis. 1987 September-October; 7(5): 463-9.
9. Kaur A., et al. Functional Skin Grafts: Where Biomaterials Meet Stem Cells. Stem Cells International. 2019. 1286054.

### Example 2. TSN Peptides in Radiation and Complex Injury Repair

In humans, doses of radiation as low as 2 Gy result in moderate radiation dermatitis, whereas levels of radiation higher than 20 Gy exposure causes radiation burns with exudation, ulceration, and sloughing of the skin [1-3]. Radiation exposure>25 Gy often involve damage to the muscle layers and bone underneath the affected skin, inhibiting normal repair processes [2]. Each year, over 4 million people in the US receive radiotherapy for the treatment of cancer, and it is estimated that 95% of these will have some level of radiation burn or radiation dermatitis [1]. While most of these cases are mild, as many as 20% may develop serious radiation dermatitis [1]. Medical treatment for radiation burns is complicated, whether they occur alone or as a component of multi-organ injuries resulting from accidental high dose radiation exposure. Repair of severe radiation burns often requires sequential surgical excisions and reconstructive surgery, or, in severe cases, amputation [1]. There are currently no Food and Drug Administration-approved agents for the prevention or mitigation of radiation-induced skin injuries [1, 2, 4, 5].

Radiation burns have pathophysiological differences from electrical or thermal burns [1, 2, 6-8]. Radiation, electrical, and thermal burns all exhibit erythema, dry or moist desquamation, ulceration, and necrosis [2, 9, 10]. However, a defining characteristic of severe radiation burns is the occurrence of unpredictable cycles of inflammation that extend the initial damage, increasing the affected area of superficial epidermis and deeper tissues, as well as delayed tissue necrosis and repair failure [2, 11]. Inflammation following ionizing radiation exposure has been shown to originate from a variety of sources. Ionizing radiation directly induces acute degranulation of mast cells in the skin [8, 12]. Additionally, radiation injury to a number of other cell types in the skin causes the release of a wide variety of inflammatory factors and cytokines [1, 13, 14]. In the skin, dendritic cells, endothelial cells, mast cells, fibroblasts and T cells are hypothesized to contribute to post-irradiation inflammation [1]. Endothelial barrier damage, that can allow vascular leak, has been demonstrated following exposure radiation at even extremely low doses, resulting in unimpeded entry of inflammatory cells into the underlying tissues as well as initiating further inflammatory reactions [15, 16]. Although direct DNA damage is usually considered to be the primary mechanism of radiation-induced injury, the activation of inflammatory pathways can initiate new rounds of DNA damage, augmenting the area of initial damage and initiating cascades of subsequent injury [17, 18].

Our research team has documented the time course of radiation-induced alterations in skin structure and cellularity in C57BL/6 mice exposed to 14-17 (0.77 Gy/min) Gy X-ray irradiation [19]. Exposure to radiation activates a variety of mechanisms inducing loss of cellular replicative potential, including necrosis, necroptosis, apoptosis, and autophagy, as well as accelerated senescence, also known as stress-induced premature senescence (SIPS) [20-24]. Results from our laboratory and others suggest that accelerated senescence may be the dominant response of normal, non-transformed, non-immortalized cells to ionizing radiation [20, 24, 25]. Extensive levels of cell death and/or senescence compromises normal tissue function. Additionally, senescent cells, although they are technically still alive, display alterations in biological activities and interactions with the surrounding tissue, such as: (1) aberrant expression of cell cycle regulatory proteins; (2) up-regulation of anti-apoptotic proteins, preventing their removal by normal processes; 3) expression of abnormal extracellular matrix proteins; and (4) robust expression of inflammatory cytokines and proteases [23, 26, 27]. This last characteristic, termed the “senescence secretory phenotype”, renders senescent cells a potent source of persistent inflammation. A recent report demonstrated that the inhibition of radiation-induced stem cell senescence in vivo in the submucosa using rapamycin reduced radiation-induced mucositis following head and neck irradiation [25]. This suggests that there is a mechanistic link between senescence, inflammation, and loss of normal repair processes.

Repair failure in radiation-induced skin injury is believed to involve the loss of replicative capacity in adult stem and progenitor cells [28]. The loss of functional adult skin stem cells (through cell death or senescence) is also thought to play a key role in skin graft failure following radiation burn injuries. In support of this hypothesis, the use of autologous mesenchymal stem cells derived from the bone marrow, to replace adult stem cells, has been utilized as a successful strategy for obtaining skin grafts for severe radiation burns [29, 30]. Additionally, another report demonstrated that inhibition of radiation-induced keratinocyte stem cell senescence in the submucosa using rapamycin was sufficient to inhibit radiation-induced mucositis [25]. This suggests that inhibition of stem cell senescence is sufficient to promote tissue repair. It is hypothesized that repair failure in radiation skin injury is due to the loss of adult stem and progenitor cells [28]. Autologous mesenchymal stem cells derived from the bone marrow have been utilized to improve survival of skin grafts for severe radiation burns [29, 30].

### Murine Model and Methods

The C57BL/6 strain of mice has been utilized extensively for the study of radiation-induced skin injuries [31]. We determined that 16 Gy is the minimal dosage for the induction of radiation burns in C57BL/6 mice that are not repaired within 45 days [19]. Sham irradiated mice without wounds were used as controls for radiation dermatitis, and treated identically as irradiated mice. Wounded mice, without radiation, were used as controls for combined injury.

Animals were irradiated in the RS2000 small animal irradiator. The RS2000 provides a cone-shaped radiation field with irradiation at 12.39-40.50 cm from the X-ray source. For dose measurements, irradiation was performed with the following settings: 160 kVp, 25 mA, 90 s irradiation time and 0.3 mm Cu beam filtration. The approximate HVL provided by the manufacturer was 0.62 mm Cu. The University of Wisconsin Medical Radiation Research Center (UW MRRC) provided eight acrylic mouse phantoms with three (1×1×1 mm) Harshaw thermoluminenscence dosimeter (TLD)-100 microcubes (ThermoElectron Corp., Oakwood Village, OH) embedded in each phantom. The cylindrical phantom had dimensions of 27 mm in diameter (D)×65 mm in length (L) and was stabilized by a cylindrical insert 15 mm (D)×27 mm (L) with a 3 mm thick stand. A custom lead shield was placed on the floor of the irradiator, with the mouse thoracic region ˜43.75 cm from the X-ray source. Dose measurements were conducted twice to account for set-up error and reproducibility. TLDs were processed at UW MRRC using a national standard with an expanded uncertainty (k=2) of 5%. The dose rate was reported for each aperture position 1-4 as an absorbed dose rate to water (ADRW, Gy/min) and the two repeated measurements were averaged. The average ADRW at any position was 0.775 Gy/min, and with 97% uniformity and with TLD measurement uncertainty it had an expanded uncertainty of +5.2%.

Mice (12-14 weeks of age) were anesthetized using inhaled anesthesia (isoflurane), and the fur was clipped on the dorsal side of the thoracic region. Two days after fur clipping, mice to be used for combined injury were given acetaminophen (150 mg/kg in 0.5 ml sterile saline) by gavage. Animals were then anesthetized with injected anesthesia (150 mg/kg ketamine/18 mg/kg xylazine) and placed in Lucite jigs for exposure to 16.5 Gy thoracic irradiation in the RS2000 X-ray irradiator as previously described [19]. For combined injury, immediately following thoracic irradiation and while still under anesthesia, the area of the skin was scrubbed with betadine or chlorhexidine, followed by cleaning with an alcohol pad. A 9/16 inch diameter punch biopsy tool was used to create a 1 cm wound in the central dorsal thoracic region, roughly in the center of the field of radiation exposure. The panniculus carnosus muscle and overlying skin will be removed. The wound will be left open to air. The size of the wound approximates 15% of the total body skin surface.

TSN6 peptide (5 μg/day in 50 μl sterile saline) or hepatocyte growth factor (HGF; 3.3 μg/day in 50 μl sterile saline) or vehicle (sterile saline, 50 μl) were injected subcutaneously on days 2, 7 and 10 post-irradiation. Injections were made caudal, adjacent to the irradiated or irradiated/wounded area.

Animals were monitored daily for 85 days post-irradiation for both radiation dermatitis and combined injury models. Radiation dermatitis was separately scored for hair loss, erythema, scale, and ulceration. Scoring for erythema, scale and ulceration were previously described: 0=none; 1=minimal; 2=mild; 3=moderate; 4=marked [19]. Because all animals underwent fur clipping prior to irradiation and wounding, scoring for hair loss was determined as failure to regrow hair in the irradiated areas: 0=none; 1=minimal; 2=mild; 3=moderate; 4=marked. Animals were photographed for the measurement of wounds and for dermatitis scoring. Animals displaying radiation-induced dermatitis will receive dermal applications of Silvadene cream (silver sulfadiazine), which is approved for human use; we have found that Silvadene can prevent infection but does not induce significant repair of radiation dermatitis or wounds. Weight was measured as an indication of overall health.

### Results

Effects of the TSN6 Peptide on Radiation Dermatitis in a Murine Model. Santyl® ointment contains a mixture of Clostridial collagenases and proteases, and is used as an enzymatic debridement agent [32, 33]. Studies by Dr. I. M. Herman showed that cellular extracellular matrix proteins digested with Santyl® ointment resulted in the release of a variety of peptides with biological activities [32, 33]. The isolated peptides were demonstrated to variably induce cell migration, proliferation, and/or angiogenesis [32, 33]. TSN6 is a 19 amino acid peptide derived from multimerin-1 upon digestion of the extracellular matrix with Santyl® [32].

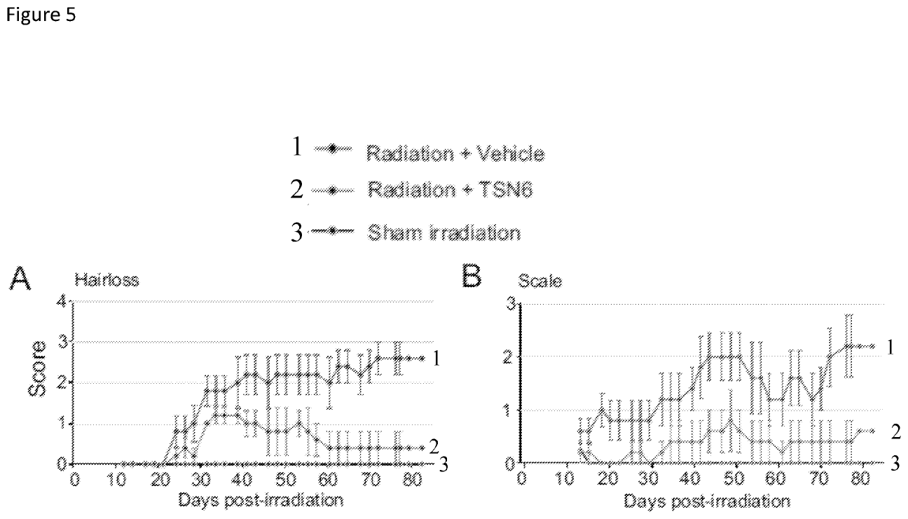

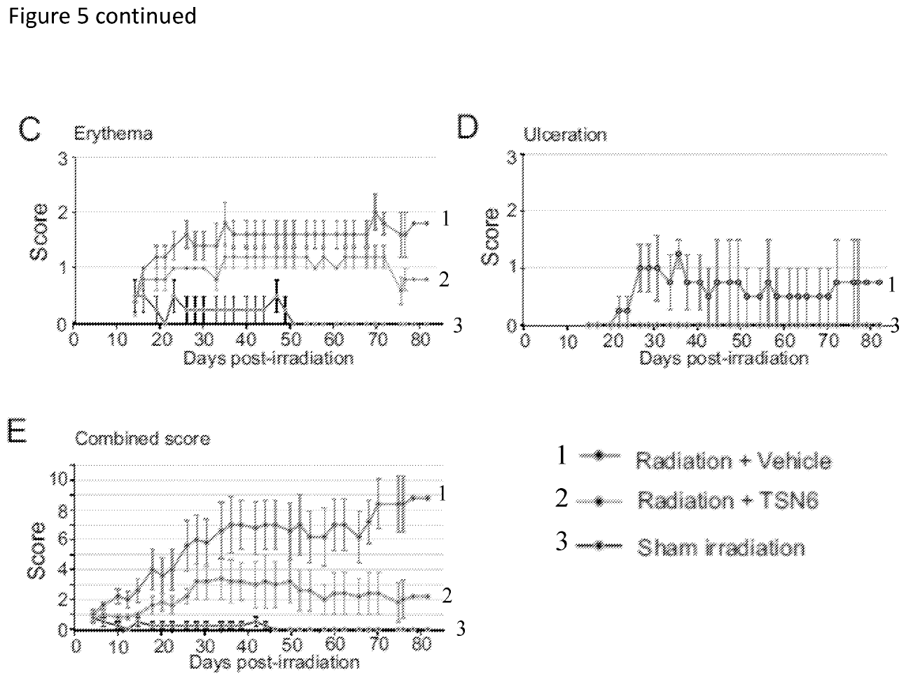

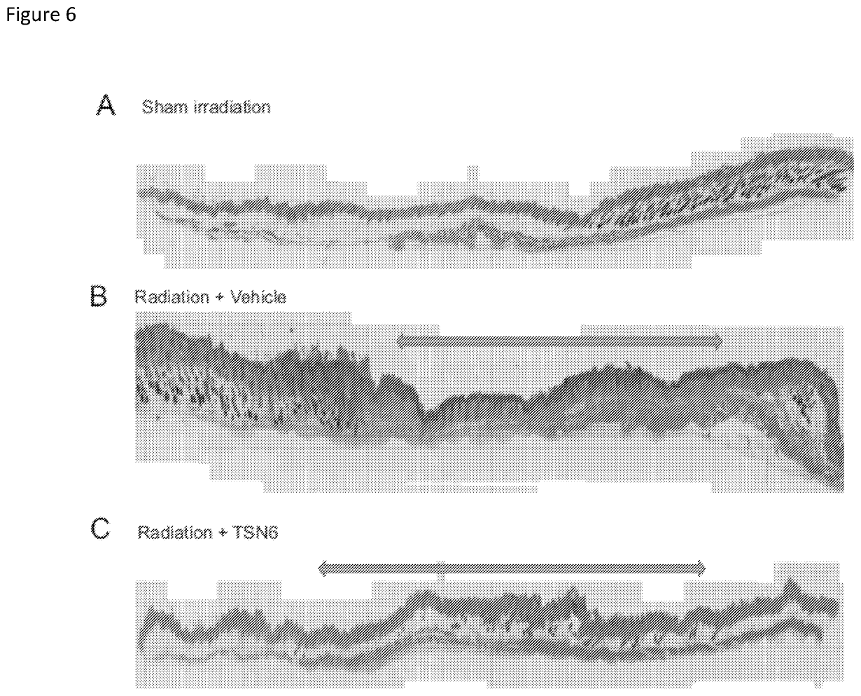

Treatment of X-ray irradiated mice with TSN6 on days 2, 7, and 10 post-irradiation mitigated radiation-induced hair loss, scale, erythema, and dermatitis compared with vehicle treatment (FIG. 5A-D). The combined scores are shown in FIG. 5E, show a marked reduction in radiation skin effects. Hematoxylin & eosin (H&E) stained histological sections indicate that radiation induced a loss of underlying adipocytes with a thickening of the overlying epidermis (FIG. 6A, B). The adipocyte layer is replaced with fibrotic tissue and inflammatory cells. Focal ulceration can be observed with a loss of the epidermal layer. Treatment with TSN6 resulted in a preservation of the adipose layer, and a reduction of the epidermal thickening (FIG. 6C). Additionally, restoration of hair follicles can be observed within the irradiated area.

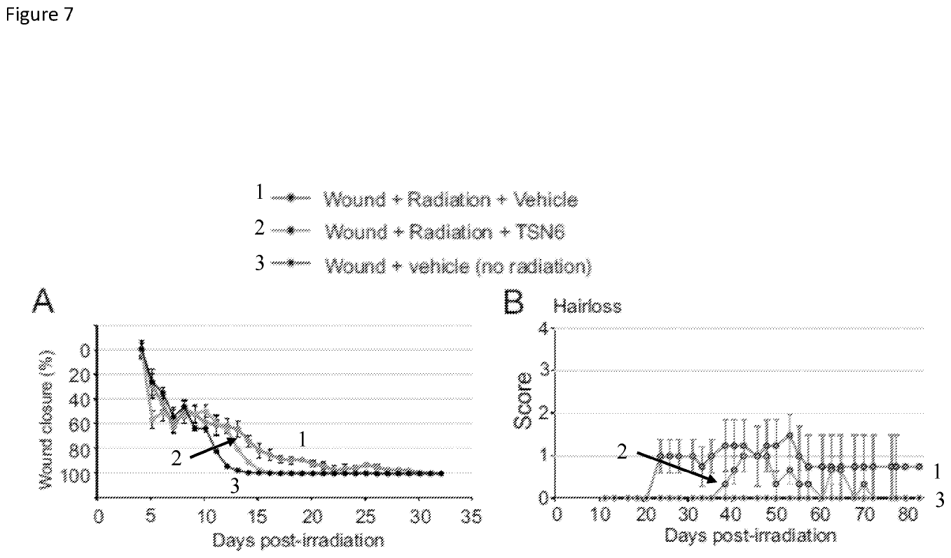

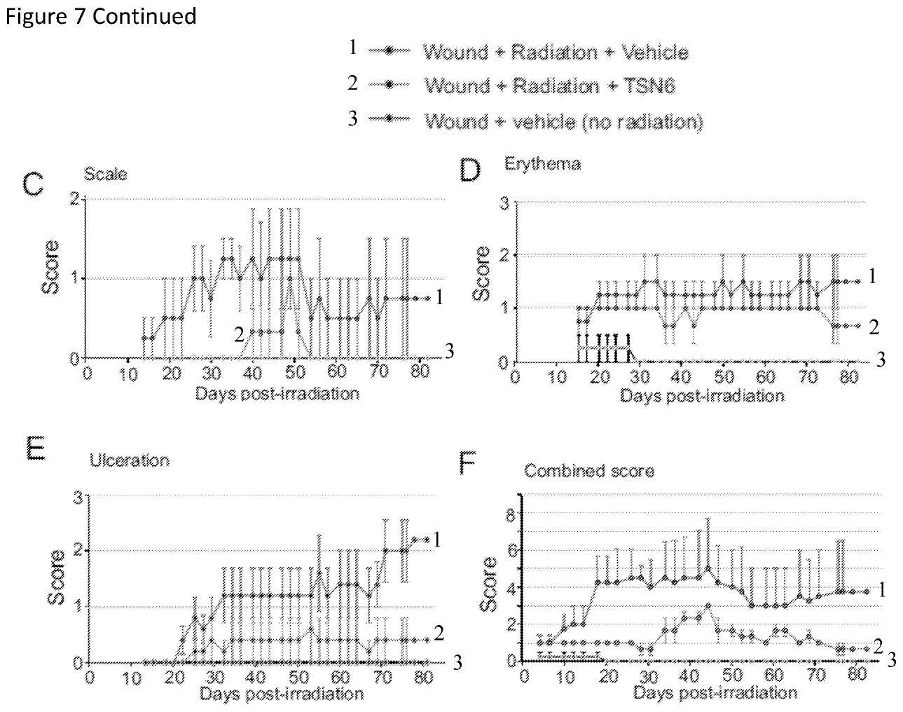

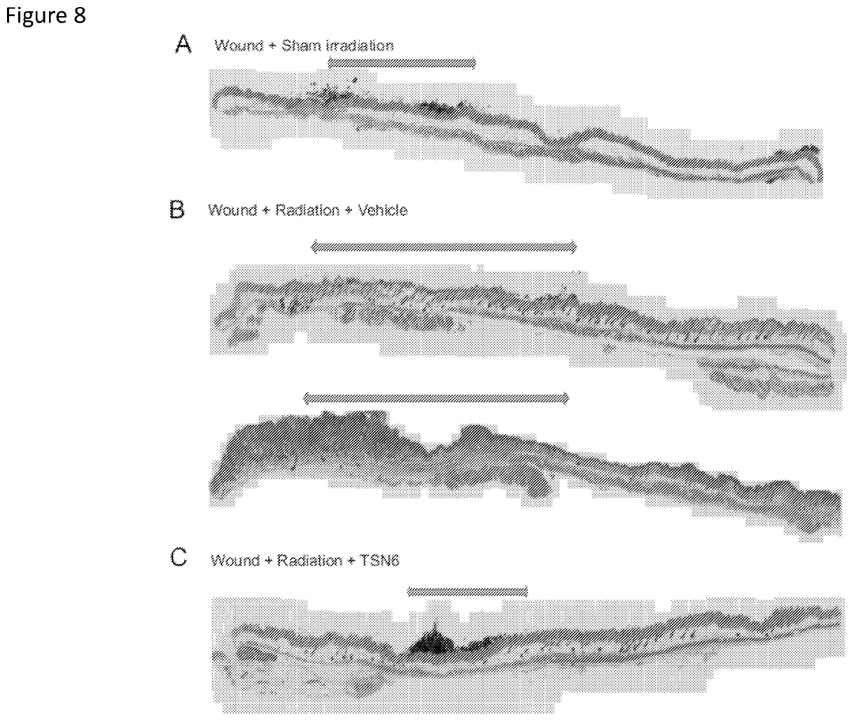

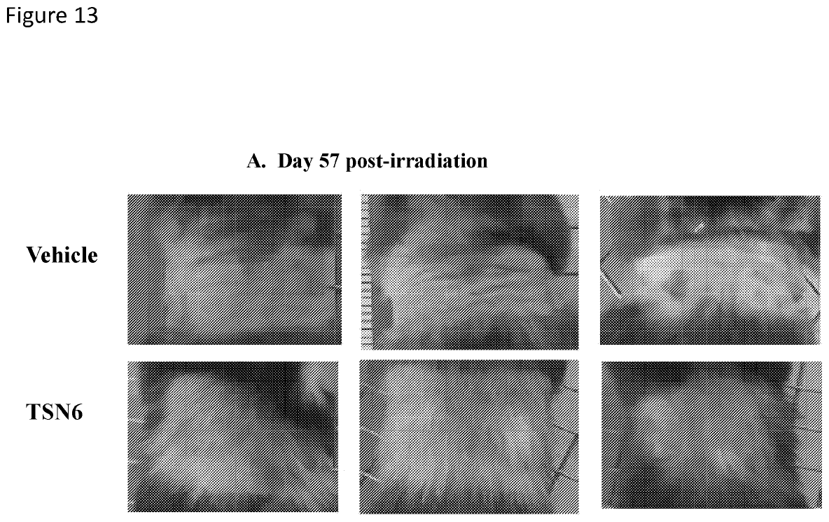

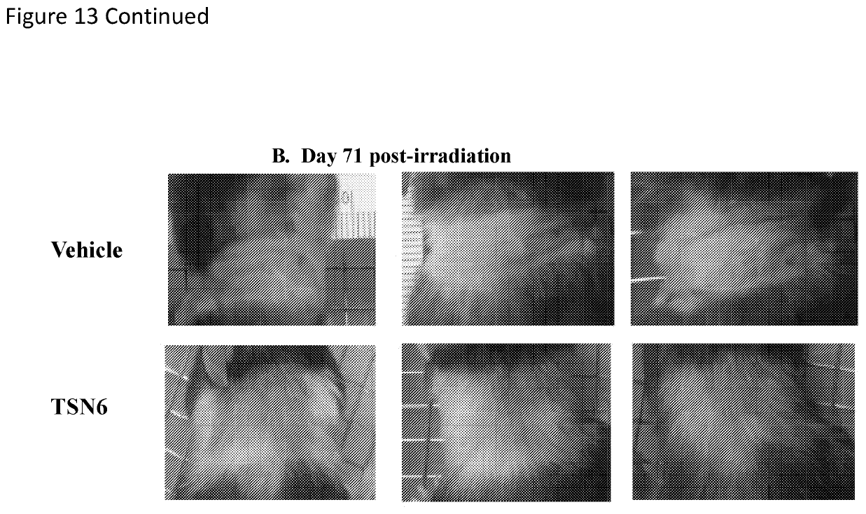

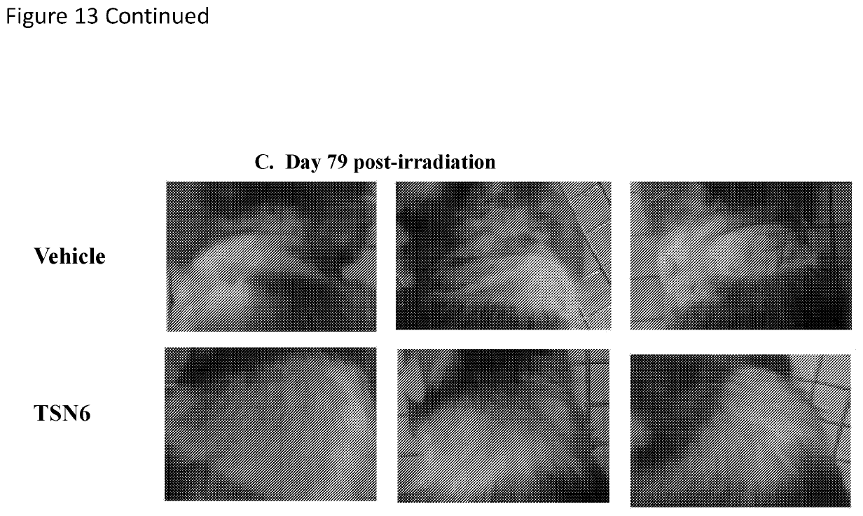

We investigated the effects of TSN6 on wound repair in the presence of radiation (termed “combined injury”). Wounds within the field of radiation typically display delayed or impaired repair capacity. Wound closure in the absence of radiation was found to be completed within ˜14 days (FIG. 7A). In contrast, radiation delayed full closure of the wounds until ˜30 days post-irradiation. TSN6 treatment increased the rate of wound closure in the presence of radiation to about 16 days post-irradiation. Hair loss, scale, erythema, and ulceration were scored in the combined injury model (FIG. 7B-F). TSN6 treatment markedly reduced all characteristics of radiation dermatitis, including in the combined score. H&E stained histological sections indicate that radiation induced a loss of underlying adipocytes with a thickening of the overlying epidermis, consistent with findings for radiation alone (FIG. 8A, B). FIG. 13A-C show vast improvement in radiation dermatitis in mice treated with TSN6 57, 71, and 75 days post-irradiation. 79 days pot-irradiation, vehicle treated mice have profound scale, erythema, ulceration and no hair regrowth, while TSN6 treated mice have full wound closure and hair regrowth (FIG. 13C).

### Effects of Hepatocyte Growth Factor on Radiation Dermatitis in a Murine Model.

Hepatocyte growth factor (HGF) is a pluripotent factor that induces cellular proliferation, survival, migration, and morphogenesis depending upon the receptive cell type [34]. HGF is required for normal development, but in the adult, HGF is required for normal tissue repair [35, 36]. HGF is a potent mitogen in human melanocytes and keratinocytes [38]. Dermal fibroblasts are the primary source of HGF in the skin although UV irradiation can trigger the expression of HGF in keratinocytes [39]. Likewise, maintaining the stability of proteases and inhibitors of the c-Met/HGF pathway is essential for optimal skin repair [35]. In skin wound repair, HGF has been demonstrated to coordinate and facilitate re-epithelialization, angiogenesis, and granulation tissue formation [35, 40, 41]. A study using the diabetic mice model, Yoshida et al. demonstrated that administration of HGF stimulates wound healing with less scarring and has a therapeutic effect on tissue fibrosis in the tight skin mouse which serves a model for of systemic sclerosis [42].

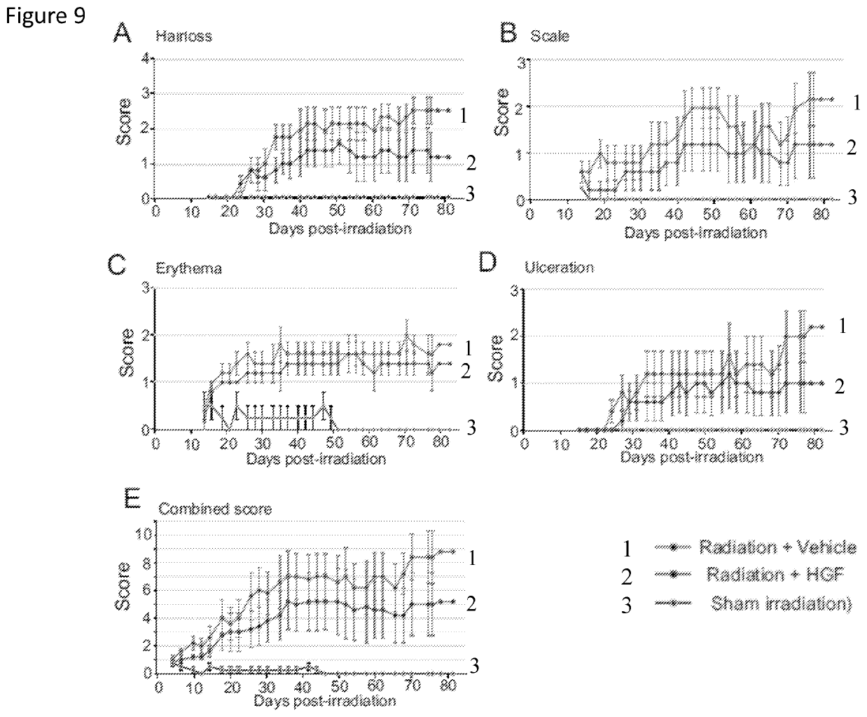

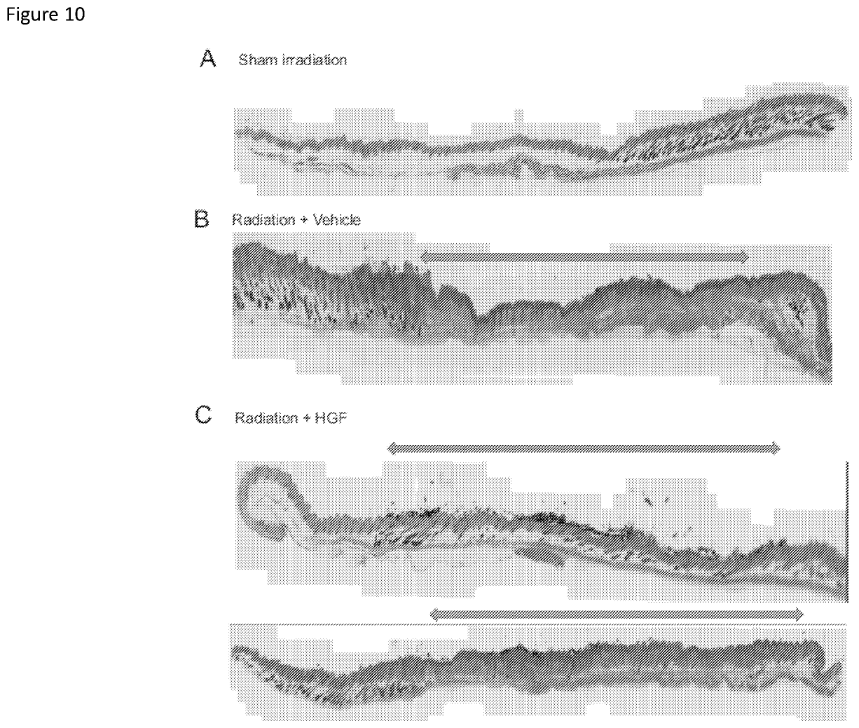

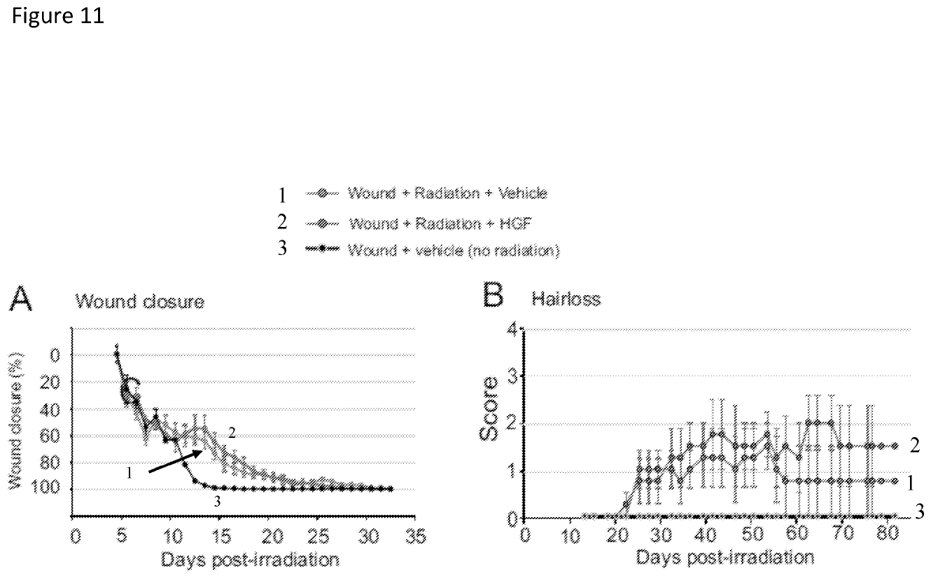

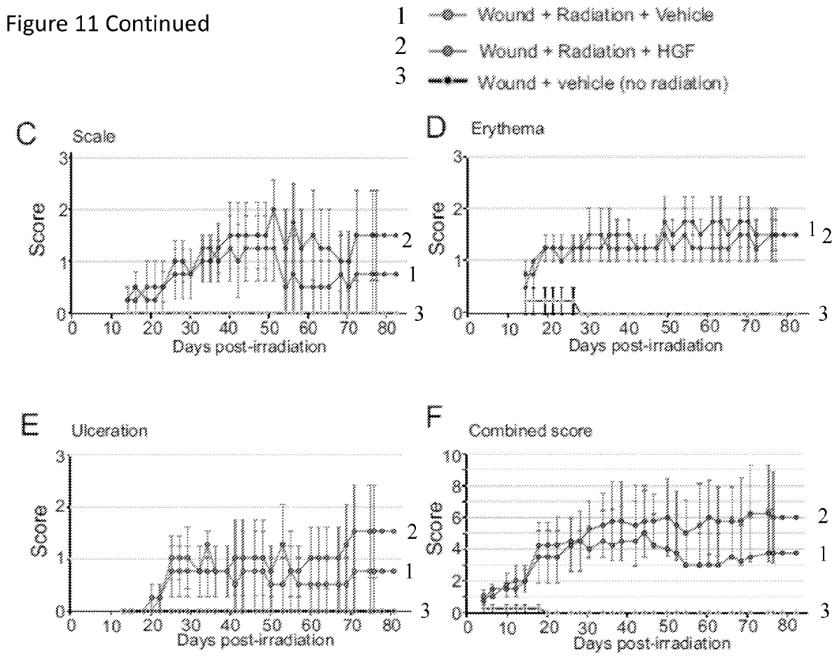

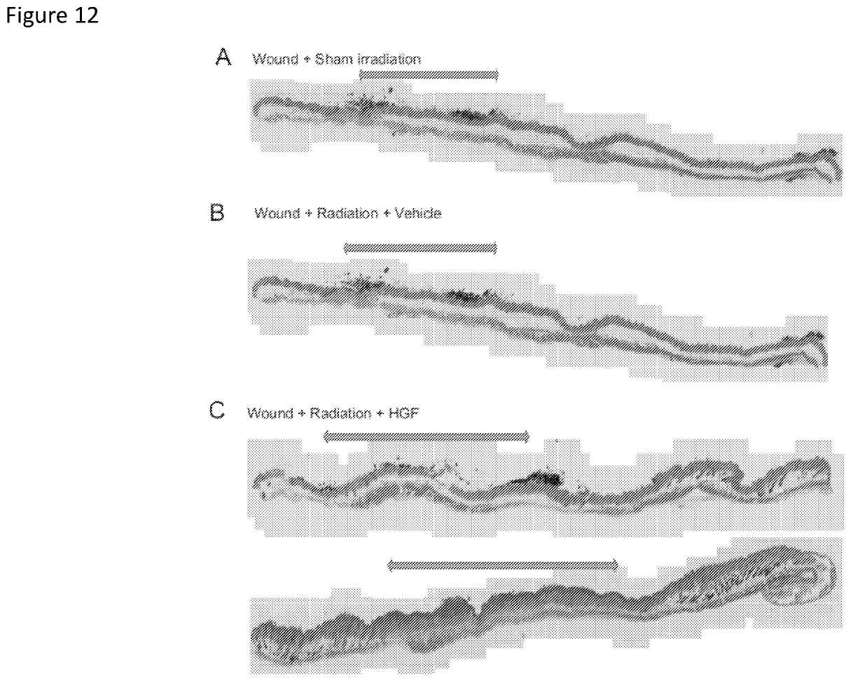

Although HGF has been shown to be effective in a variety of wound models, HGF did not improve any measures of radiation dermatitis (FIG. 9). Hair loss, scale, erythema, and ulceration were not markedly different between vehicle and HGF-treated animals. Histological sections did show some improve preservation of the adipose layer in HGF-treated animals, but this effect appeared to be inconsistent within the group (FIG. 10). Additionally, HGF did not accelerate wound repair in the combined injury model (FIG. 11A). In the combined injury model, HGF treatment did not improve any measure of radiation dermatitis, and seemed to somewhat exacerbate ulceration (FIG. 11B-F). Histological sections of the combined injury did show some improvement in the adipose layer in some animals, but as for radiation alone, this effect was not consistent within the group (FIG. 12).

Together our data indicate that mitigation of radiation dermatitis and combined injury are a unique property of TSN6 peptide administration. These biological activities are not recapitulated by a known adult tissue repair factor, HGF.

### REFERENCES EXAMPLE 2

1. Ryan, J. L., Ionizing radiation: the good, the bad, and the ugly. J Invest Dermatol, 2012. 132(3 Pt 2): p. 985-93.
2. Benderitter, M., et al., New emerging concepts in the medical management of local radiation injury. Health Phys, 2010. 98(6): p. 851-7.
3. Giordano, S., Radiation-induced skin injuries during interventional radiography procedures. J Radiol Nurs, 2010. 29: p. 37-47.
4. Hoashi, T., et al., A case of acute radiation syndrome from the dermatological aspect. Br J Dermatol, 2008. 158(3): p. 597-602.
5. Ryan Wolf, J., et al., Utility of topical agents for radiation dermatitis and pain: a randomized clinical trial. Support Care Cancer, 2020. 28(7): p. 3303-3311.
6. Hopewell, J. W., The skin: its structure and response to ionizing radiation. Int J Radiat Biol, 1990. 57(4): p. 751-73.
7. Rifkin, L. H., et al., An athymic rat model of cutaneous radiation injury designed to study human tissue-based wound therapy. Radiat Oncol, 2012. 7: p. 68.
8. Albrecht, M., et al., Ionizing radiation induces degranulation of human mast cells and release of tryptase. Int J Radiat Biol, 2007. 83(8): p. 535-41.
9. Li, Y., et al., Successful Treatment of a Case of Extensive Radiation Burns With Multiple Organ Dysfunction Syndrome. J Burn Care Res, 2013. 34: p. e014-9.
10. Koenig, T. R., F. A. Mettler, and L. K. Wagner, Skin injuries from fluoroscopically guided procedures: part 2, review of 73 cases and recommendations for minimizing dose delivered to patient. AJR Am J Roentgenol, 2001. 177(1): p. 13-20.
11. Simard, P. F., R. M. Bolton, and N. J. Tarbell, Anti-inflammatory cream reduces skin damage induced by ionizing radiation. Oncologist, 2009. 14(2): p. 197-8.
12. Rabenhorst, A., et al., Mast cells play a protumorigenic role in primary cutaneous lymphoma. Blood, 2012.
13. Kuilman, T. and D. S. Peeper, Senescence-messaging secretome: SMS-ing cellular stress. Nat Rev Cancer, 2009. 9(2): p. 81-94.
14. Liang, L., et al., Celecoxib reduces skin damage after radiation: selective reduction of chemokine and receptor mRNA expression in irradiated skin but not in irradiated mammary tumor. Am J Clin Oncol, 2003. 26(4): p. S114-21.
15. Griem, M. L., A. Robotewskyj, and R. H. Nagel, Potential vascular damage from radiation in the space environment. Adv Space Res, 1994. 14(10): p. 555-63.
16. Frazier, T. H., et al., Fluoroscopy-induced chronic radiation skin injury: a disease perhaps often overlooked. Arch Dermatol, 2007. 143(5): p. 637-40.
17. Hill, R. P., et al., Investigations into the role of inflammation in normal tissue response to irradiation. Radiother Oncol, 2011. 101(1): p. 73-9.
18. Calveley, V. L., et al., Partial volume rat lung irradiation: temporal fluctuations of in-field and out-of-field DNA damage and inflammatory cytokines following irradiation. Int J Radiat Biol, 2005. 81(12): p. 887-99.
19. McCart, E. A., et al., Accelerated senescence in skin in a murine model of radiation-induced multi-organ injury. J Radiat Res, 2017. 58(5): p. 636-646.
20. Panganiban, R. A. and R. M. Day, Inhibition of IGF-IR prevents ionizing radiation-induced primary endothelial cell senescence. PLOS One, 2013. 8(10): p. e78589.
21. Panganiban, R. A., A. L. Snow, and R. M. Day, Mechanisms of radiation toxicity in transformed and non-transformed cells. Int J Mol Sci, 2013. 14(8): p. 15931-58.
22. Kaliberov, S. A. and D. J. Buchsbaum, Chapter seven—Cancer treatment with gene therapy and radiation therapy. Adv Cancer Res, 2012. 115: p. 221-63.
23. Suzuki, M. and D. A. Boothman, Stress-induced premature senescence (SIPS)-influence of SIPS on radiotherapy. J Radiat Res, 2008. 49(2): p. 105-12.
24. Panganiban, R. A., O. Mungunsukh, and R. M. Day, X-irradiation induces ER stress, apoptosis, and senescence in pulmonary artery endothelial cells. Int J Radiat Biol, 2013. 89(8): p. 656-67.
25. Iglesias-Bartolome, R., et al., mTOR Inhibition Prevents Epithelial Stem Cell Senescence and Protects from Radiation-Induced Mucositis. Cell Stem Cell, 2012. 11(3): p. 401-14.
26. Muller, M., Cellular senescence: molecular mechanisms, in vivo significance, and redox considerations. Antioxid Redox Signal, 2009. 11(1): p. 59-98.

27 Tchkonia, T., et al., Cellular senescence and the senescent secretory phenotype: therapeutic opportunities. J Clin Invest, 2013. 123(3): p. 966-72.
28. Ahmed, E. A., et al., Persistent DNA damage after high dose in vivo gamma exposure of minipig skin. PLOS One, 2012. 7(6): p. e39521.
29. Bey, E., et al., Emerging therapy for improving wound repair of severe radiation burns using local bone marrow-derived stem cell administrations. Wound Repair Regen, 2010. 18(1): p. 50-8.
30. Agay, D., et al., Multipotent mesenchymal stem cell grafting to treat cutaneous radiation syndrome: development of a new minipig model. Exp Hematol, 2010. 38(10): p. 945-56.
31. Haston, C. K., Mouse genetic approaches applied to the normal tissue radiation response. Front Oncol, 2012. 2: p. 94.
32. Sheets, A. R., et al., Identification and Characterization of Novel Matrix-Derived Bioactive Peptides: A Role for Collagenase from Santyl® Ointment in Post-Debridement Wound Healing? PLOS One, 2016. 11(7): p. e0159598.
33. Sheets, A. R., et al., Matrix- and plasma-derived peptides promote tissue-specific injury responses and wound healing in diabetic swine. J Transl Med, 2016. 14(1): p. 197.
34. Rubin, J. S., D. P. Bottaro, and S. A. Aaronson, Hepatocyte growth factor scatter factor and its receptor, the c-met proto-oncogene product. Biochim Biophys Acta, 1993. 1155(3): p. 357-71.
35. Buchstein, N., et al., Alternative proteolytic processing of hepatocyte growth factor during wound repair. Am J Pathol, 2009. 174(6): p. 2116-28.
36. Yoshida, S., et al., Recombinant hepatocyte growth factor accelerates cutaneous wound healing in a diabetic mouse model. Growth Factors, 2004. 22(2): p. 111-9.
37. Matsumoto, K., H. Tajima, and T. Nakamura, Hepatocyte growth factor is a potent stimulator of human melanocyte DNA synthesis and growth. Biochem Biophys Res Commun, 1991. 176(1): p. 45-51.
38. Matsumoto, K., et al., Marked stimulation of growth and motility of human keratinocytes by hepatocyte growth factor. Exp Cell Res, 1991. 196(1): p. 114-20.
39. Mildner, M., et al., Hepatocyte growth factor establishes autocrine and paracrine feedback loops for the protection of skin cells after UV irradiation. J Invest Dermatol, 2007. 127(11): p. 2637-44.
40. Toyoda, M., et al., Overexpression of hepatocyte growth factor scatter factor promotes vascularization and granulation tissue formation in vivo. FEBS Lett, 2001. 509(1): p. 95-100.
41. Bevan, D., et al., Diverse and potent activities of HGF SF in skin wound repair. J Pathol, 2004. 203(3): p. 831-8.
42. Iwasaki, T., et al., Hepatocyte growth factor ameliorates dermal sclerosis in the tight-skin mouse model of scleroderma. Arthritis Res Ther, 2006. 8(6): p. R161

### Example 3. Use of Next Generation Bioactive Peptides in Skin Substitutes to Promote Wound Healing and Skin Regeneration

Introduction: Acute and chronic wounds affect more than 6 million people annually in the United States alone, and the repair and management of full thickness wounds has been a long standing challenge. Tissue engineered skin substitutes are used in the clinic to treat wounds. One type of skin substitute, dermal-epidermal composites (DEC), also known as skin equivalents or bilayered living skin constructs, are comprised of dermal fibroblasts embedded in a matrix such as collagen and overlaid with keratinocytes. DECs promote wound healing and have been used to model skin development and diseases. Potential limitations of skin substitutes include delayed vascularization, contracture, loss of sensitivity, abnormal pigmentation, and absence of hair follicles (HFs) and sebaceous glands which can lead to impaired healing and scarring. The overall goal of our skin research program is to increase our understanding of wound healing and skin regeneration and to develop products that improve the function of skin and promote scarless healing. Our quest to develop innovative and advanced therapeutics to treat wounds, accelerate healing and improve graft outcomes lead us to investigate next generation, protease-resistant, pro-healing peptides which have been previously demonstrated to stimulate post-injury granulation tissue formation, neovascularization and wound re-epithelialization. These bioactive peptides were identified by Dr. Herman at Tufts University as products cleaved from native human extracellular matrix proteins by agents used for the debridement of necrotic tissue from chronic wound sites and severely burned areas. Previous studies demonstrated that TSN18, a peptide created from non-helical domains of type VI collagen, promoted wound re-epithelization and closure in vitro and in BALB/c mice. Given these results, and the growing evidence that collagen VI has critical functions in stem cells niches and is regulated by skin wounding, we explored whether TSN18 promotes skin cell viability in vitro and in vivo to improve the durability of grafted DECs.

Materials and Methods: Cultured dermal papilla (DP) cells, specialized skin fibroblasts isolated from the base of hair follicles, were treated with TSN18 for four days. Cell viability was measured using the CellTiter 96 Non-radioactive cell proliferation assay (MTT) kit. Alkaline phosphatase activity, a marker of trichogenicity was measured with the alkaline phosphatase yellow (pNPP) liquid substrate system for ELISA. A pilot study was performed to see whether incorporation of TSN18 active peptide in DECs improves graft survival or size. For the construction of DECs, human DP cells suspended in 1 mg/mL rat tail type I collagen were plated on inserts placed in six-well plates and incubated in media containing TSN18 active or scrambled peptide for 3 days. Subsequently, human neonatal foreskin keratinocytes were layered on top and the media changed to Epi medium containing TSN18 active or scrambled peptide for 2 days. The DECs were lifted to air-liquid interface for 2 more days by adding fresh Cori medium to the bottom. Cr: NIH(S)-nu/nu mice were grafted by placing the DECs on cutaneous wounds and the mice were bandaged to shield the graft. The bandages were changed at two weeks and removed after four weeks and the grafts were monitored through 10 weeks. As typical in these experiments, graft sizes gradually diminished over time. At the end of the experiment, the histological appearance of grafts was evaluated for the presence of a human stratified squamous epithelium and the presence or absence of human cells was assessed by staining for human leukocyte antigen. Graft sizes were measured by tracing the grafts and area determined using ImageJ (NIH) software.

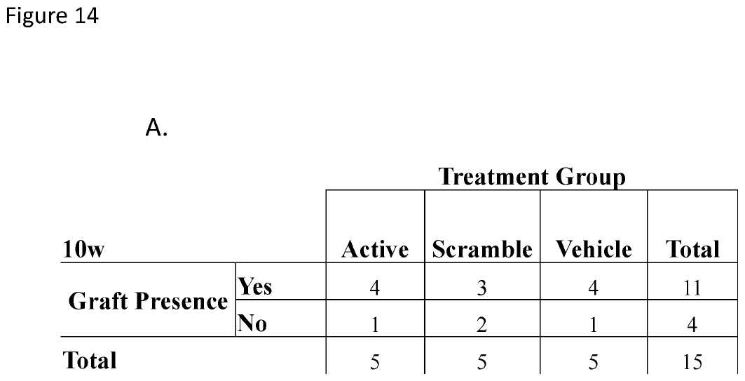

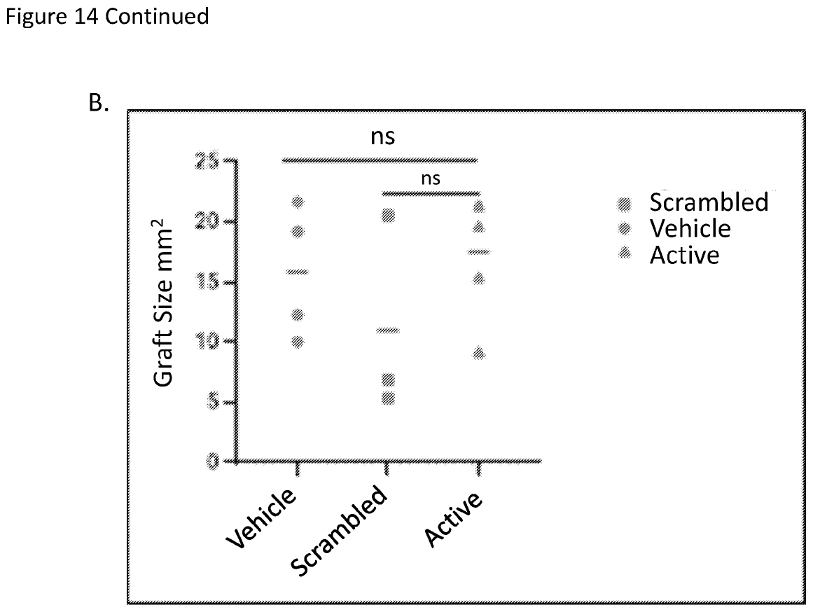

Results: TSN18 did not affect cell viability or alkaline phosphatase activity of DP cells in vitro. Results from the grafting experiments showed that four out of five mice grafted with DECs without peptide or mice grafted with DECs with TSN18 active peptide retained the graft until 10 weeks while three out of the five mice grafted with DECs with scrambled peptide had a graft at 10 weeks (FIG. 14). Average graft size determined using ImageJ (NIH) software, for vehicle, active peptide and scrambled peptide groups were 15.8±5.5 mm2, 16.4±5.4 mm2 and 10.9±8.4 mm2, respectively.

Discussion: Our data suggest that bioactive peptides can be safely incorporated into skin substitutes to promote graft durability and graft size, supporting their use in the development of next-generation skin substitutes to enhance wound healing.

### Example 4. Deploying Bioactive Peptides in Bioprinted Dermal-Epidermal Composites for Improved Skin Regeneration

Introduction: Full-thickness skin injuries are common in warfighters after traumatic blasts, leading to physical, mental and emotional distress for them and their families during and after recovery. Current methods to promote skin repair include using autologous skin grafts that require existing donor tissue transplanted to the wound site. Split-thickness grafts are commonly used but they lack components within the dermal layer needed to induce hair regeneration and restore more normal skin function. Our goal is to bioengineer dermal-epidermal composites (DECs) to promote complete skin regeneration without scarring.

DECs have been developed that are comprised of a collagen matrix embedded with fibroblasts and overlaid with human keratinocytes to enhance the proliferative and regenerative phases of wound healing. Our laboratory has demonstrated human hair follicle neogenesis in DECs by using adult human dermal papilla cells overlaid with human keratinocytes then grafted to immunodeficient mice. We have made advances in this technique by: 1) using 3D bioprinting to construct the DECs, and 2) incorporating dermal papilla spheroids into the collagen. Dermal papilla cells lose the ability to induce hair follicles during growth in monolayers, and aggregation into spheroids helps restore trichogenicity. The next phase is to combine these advances with the incorporation of protease-resistant, pro-healing peptides developed by Dr. Ira Herman at Tufts University. These peptides have been previously demonstrated to significantly stimulate post-injury granulation tissue formation, neovascularization and wound re-epithelialization, properties expected to enhance skin regeneration in DECs. We hypothesized that bioactive peptides, used in the formation of dermal papilla cell spheroids, promote hair follicle formation in the bioprinted DECs.

Materials and Methods: We tested the compatibility of human dermal papilla cells, suspended in medium plus 0.1 mg/mL rat tail type 1 collagen, with active or scrambled peptide at 1, 10 and 50 μM concentrations during spheroid formation using the hanging drop method. Six DECs were constructed with scrambled peptide, seven with active peptide, and six with vehicle containing no peptide. Briefly, human dermal papilla cell spheroids were formed by hanging drop method in the presence of 10 μM active peptide, scrambled peptide, or vehicle. Spheroids were added to rat type 1 collagen and bioprinted into transwell inserts of a six-well plate using the CellInk BioX printer. DECs were submerged in wells containing dermal papilla cell media for two days. 1,000,000 human keratinocytes were bioprinted directly onto the DECs and incubated in PRIME AIRLIFT media for two days prior to raising the DECs to the air-liquid interface for two more days. One DEC with active peptide was constructed with the same components by manual pipetting, rather than bioprinting, before grafting into a mouse.

Results: The microscopic appearance of the spheroids was maintained in hanging drops in the presence of scrambled or active peptide at all concentrations. DECs with 10 μM of active or scrambled peptides were intact and similar to vehicle after bioprinting. Bioprinted DECs were evaluated using an inverted light microscope, showing formation of a uniformly dense sheet of epithelium overlying scattered spheroids within the DEC. Spheroids maintained microscopic appearance in DECs before and after adding keratinocytes. The pipetted DEC with active peptide was successfully grafted into a mouse. Histology results display a thicker epidermal region of human keratinocytes compared to the remaining mouse epidermis.

Discussion: Human dermal papilla spheroids formed in the presence of bioactive peptide were successfully bioprinted into DECs. Spheroid microscopic appearance did not appear to be affected by the bioprinting process or inclusion of peptides. A DEC made by manual pipetting and grafted into a mouse showed graft take and presence of human cells 4 weeks after grafting. Our plan is to further optimize DECs with bioactive peptide that can be successfully bioprinted and grafted into mice as an in vivo model for skin regeneration and hair follicle growth. The goal is to further develop DECs to improve wound healing and regeneration of normal skin structures that can be translated from the bench to the warfighter.

### Example 5. The Development of Countermeasures for Cutaneous Radiation Injuries and Combined Injuries

Introduction: The Department of Defense (DOD) is prepared to execute military missions following radiation accidents, to manage radiation crises associated with terrorist activities, and to manage consequences in the event of nuclear weapons detonation. For instance, the US military was among the first responders to provide assistance to support Japan in disaster relief following the Fukushima nuclear power plant disaster in 2011 (Operation Tomodachi). Recent studies indicate that there is an increasing risk of exposure to ionizing radiation to military personnel due to increasing terrorist activities, including the potential use of stolen nuclear weapons, the detonation of an improvised nuclear device (IND), or a large radiological dispersal device (RDD). Cutaneous Radiation Injuries (CRI) are a significant cause of morbidity following radiation exposure and are a significant concern for nuclear accidents and radiation combat events. Additionally, studies estimate that in a radiation mass casualty or combat scenario, 65-70% of individuals' radiation exposure will be coupled with other injuries including wounds, a condition termed combined injury (CI). Repair failure in CRI and CI is associated with the occurrence of unpredictable cycles of inflammation, loss of adult skin stem cells, tissue necrosis, and fibrotic remodeling. Unlike thermal burns, skin grafting following CRI is effective only with co-administration of autologous stem cells, a therapy not suited for mass casualty events. Currently, there are four radiation countermeasures approved by the US Food and Drug Administration for the mitigation of acute radiation syndrome, all of which address the loss of mature white blood cells following high-dose radiation exposure. Agents appropriate for the mitigation of CRI or CI in mass casualty or combat scenarios are needed. For efficacy in a mass casualty or military combat situation, such agents should be stable, easily administered, and effective when delivered at least 48 h following injury and radiation exposure. We hypothesized that wound healing peptides, identified as protease-resistant fragments of extracellular matrix proteins by investigators at Tufts University, represent a new class of agents to test for mitigating CRI. Here we describe the development of a robust preclinical murine model for CRI and CI and promising preliminary results using bioactive peptides as a radiation countermeasure.

Materials and Methods: We used the C57BL/6 mouse strain, which displays both radiation-induced inflammation and fibrotic remodeling, similar to human responses. Mice were exposed to thoracic X-ray irradiation without excision wounding to induce CRI, and with an experimental excision wound within the irradiated region to induce CI. Animals were scored by researchers blinded to the treatment groups. Photographic documentation was made for each experiment. We evaluated the rate of wound healing from days 1-30 post-irradiation, and the severity of dermatitis was scored using four variable measures (erythema, scale, hair loss, and ulceration) in the mice from 14 days post-irradiation (dpi) until necropsy. Specific cellular and protein expression changes were determined using western blotting and immunohistochemistry (IHC). To evaluate the efficacy of bioactive peptides, mice with CI were treated with active peptides or scrambled peptides (negative control) by subcutaneous injection near the wound site on days 2, 7, and 10 post-irradiation. Additionally, vehicle control CI animals received saline (vehicle for the peptides) injections in the same volumes on the same days.

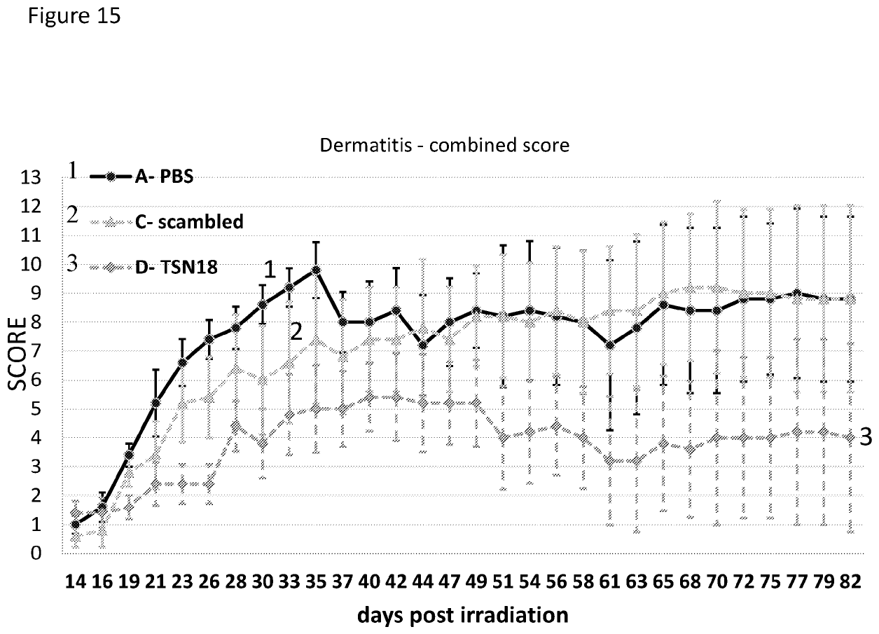

Results: In the CRI model, 14 Gy (0.77 Gy/min) irradiation resulted in mild inflammation, observed histologically, without hair loss or erythema. 16 or 17 Gy (0.77 Gy/min) radiation-induced dry desquamation, erythema, and mild ulceration, detectable within 14 dpi. Histological evaluation revealed inflammation with mast cell infiltration within 14 dpi at the higher doses of radiation. Scar formation, or fibrosis, occurred 80 days following 16 or 17 Gy (0.77 Gy/min), characterized by collagen deposition, mast cell and neutrophilic dermatitis, and necrotic debris. Using western blotting and IHC, we identified accelerated senescence at 7 dpi, and apoptosis at 30 dpi, both biological events preceding inflammation and fibrosis. Importantly, accelerated senescence occurred in cells at the base of the hair follicle bulbs, the location of the adult stem cells of the skin. In the CI model, the presence of radiation resulted in a 2-fold increase in the time required for wound closure. IHC also identified accelerated senescence in the keratinocyte and adult stem cell populations in the radiation field, similar to the CRI model. Additionally, the deposition of collagen was significantly altered in CI compared with wound without radiation. Our pilot study using 5 mice per group, showed that injection of active but not scrambled peptide decreased the severity of our aggregate radiation dermatitis scores of the mouse skin by nearly 50% (FIG. 15).

Discussion: Our data indicate that our C57BL/6 murine models for CRI and CI recapitulate many of the characteristics observed in humans following high-dose radiation injury to the skin, including the loss of adult stem cells, the activation of several types of inflammatory cells, and exaggerated fibrotic remodeling. Additionally, our data indicate that the combination of radiation in the presence of a wound results in delayed wound healing, also as observed in humans with these injuries. Importantly, the adult stem cells of the skin are a cell population required for normal tissue regeneration, and senescence of this population may underlie repair failure following radiation exposure. Our data also indicate that Tufts bioactive peptides reduce the development of radiation-induced dermatitis when administered in a delayed time course. Therefore, these peptides are a promising new radiation countermeasure that could be administered following a combat or mass casualty event.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

1. A method for treating a subject having hair-loss, or a subject at risk for hair loss or progression of hair loss, or a subject with impaired function or loss of hair or other skin adnexal structures, the method comprising administering to the subject an effective amount of a peptide comprising the amino acid sequence of SEQ ID NO:6, any one of SEQ ID NOs: 1-5, 7-19, or combinations thereof, for treating the hair-loss, or stimulating the formation, preservation, or function of hair follicles or other skin adnexa.

2-4. (canceled)

5. The method of claim 1, wherein the peptide consists of a combinatorial peptide comprising one or more SEQ ID NOs: 1-19 fused to a second peptide or polypeptide.

6. (canceled)

7. The method of claim 1, where in the peptide is combined with a carrier or excipient or transdermal delivery system, optionally wherein the peptide is combined with follicular units or a substrate prior to administering the peptide to the subject, optionally via transplantation.

8. The method of claim 1, wherein the peptide is administered locally to the treatment area, optionally wherein the peptide is administered ex vivo to cells, a substrate, or engineered skin prior to administration to the patient.

9. The method of claim 1, wherein the effective amount of the peptide is effective for promoting neogenesis or preservation of hair follicles or improvement in hair follicle function.

10-14. (canceled)

15. The method of claim 1, wherein the subject has hair-loss attributed to the loss of hair follicles.

16. (canceled)

17. The method of claim 1, wherein the subject has hair-loss attributed to androgenetic alopecia.

18. The method of claim 1, wherein the subject has hair-loss due to trauma, and optionally physical trauma, thermal trauma, chemical trauma, or radiological trauma or other forms of trauma.

19. The method of claim 18, wherein the trauma results in full or partial skin loss and/or reduction in skin thickness.

20. The method of claim 1, wherein the subject has a scarring form of alopecia optionally selected from central centrifugal cicatricial alopecia, lichen planopilaris, acne keloidalis nuchae, dissecting cellulitis, traction alopecia, and pseudopelade of Brocq.

21. The method of claim 1, wherein the subject has a non-scarring form of alopecia, optionally selected from alopecia areata, anagen effluvium, and telogen effluvium.

22. The method of claim 1, wherein the subject has hair-loss attributed to a burn wound.

23. The method of claim 1, wherein the subject has hair-loss attributed to a treatment that results in the death of hair follicles.

24. The method of claim 1, wherein the subject has hair-loss attributed to radiation treatment or chemotherapy treatment.

25. (canceled)

26. A method for mitigating the effects of radiation therapy in a subject undergoing radiation therapy, the method comprising administering to the subject an effective amount of an effective amount of a peptide comprising the amino acid sequence of SEQ ID NO: 18 or any one of SEQ ID NOs: 1-17 or SEQ ID NO: 19 for mitigating the effects of radiation therapy.

27-29. (canceled)

30. The method of claim 2926, wherein the peptide consists of a combinatorial peptide comprising one or more SEQ ID NOs: 1-19 fused to a second peptide or polypeptide.

31. (canceled)

32. The method of claim 26, where in the peptide is combined with a carrier or excipient or transdermal delivery system.

33-36. (canceled)

37. The method of claim 26, wherein the effective amount of the peptide is effective for treating dermatitis.

38. The method of claim 26, wherein the effective amount of the peptide is effective for mitigating stem cell senescence in the skin.

39. The method of claim 26, wherein the effective amount of the peptide is effective for promoting neogenesis or preservation of hair follicles or hair follicle function.

40-43. (canceled)
